(19) United States
(12) Patent Application Publication
(10) Pub. No.: US 2026/0060979 A1
(43) Pub. Date: Mar. 5, 2026

(54) METHOD FOR IMPROVING NEUROPLASTICITY

(71) Applicant: Alto Neuroscience, Inc., Mountain View, CA (US)

(72) Inventors: Amit Etkin, Mountain View, CA (US); Adam Savitz, Mountain View, CA (US); Akash Datwani, Mountain View, CA (US); Maria Ferreira da Silva, Mountain View, CA (US)

(21) Appl. No.: 19/316,824

(22) Filed: Sep. 2, 2025

Related U.S. Application Data
(60) Provisional application No. 63/690,534, filed on Sep. 4, 2024.
(60) Provisional application No. 63/690,541, filed on Sep. 4, 2024.
(60) Provisional application No. 63/701,656, filed on Oct. 1, 2024.
(60) Provisional application No. 63/701,641, filed on Oct. 1, 2024.
(60) Provisional application No. 63/701,644, filed on Oct. 1, 2024.

(51) Int. Cl. A61K 31/496; A61K 31/137; A61K 31/55; A61P 25/00; A61P 25/24

(52) U.S. Cl. CPC A61K 31/496; A61K 31/137; A61K 31/55; A61P 25/00; A61P 25/24

(57) ABSTRACT

This invention relates to the use of (i) a 5-HT_{2A} agonist (including 5-HT_{2A} agonists that bind to a BDNF receptor, e.g., a 5-HT_{2A} agonist that binds to the TrkB receptor or p75NTR), (ii) a therapeutic agent that promotes neuroplasticity by stimulation of the 5-HT_{2A} receptor, (iii) a therapeutic agent that releases BDNF by stimulation of the 5-HT_{2A} receptor (including those therapeutic agents that stimulate the 5-HT_{2A} receptor and bind to a TrkB receptor or p75NTR), (iv) an AMPA positive allosteric modulator, (v) a therapeutic agent that promotes neuroplasticity by stimulation of the AMPA receptor, (vi) a therapeutic agent that releases BDNF by stimulation of the AMPA receptor, or (vii) an NMDA receptor positive allosteric modulator (such as a stinel compound) in the treatment of a psychiatric condition in which depressive symptoms are prominent, including major depressive disorder (MDD), bipolar disorder, post-traumatic stress disorder, substance use disorder, and depression-related aspects of schizophrenia (e.g. negative symptoms) in select patients who, for instance, have objectively determined cognitive impairment or poor cognition (such as objectively determined impaired learning and/or memory) and/or certain EEG characteristics.

This application claims the benefit of (i) U.S. Provisional Application No. 63/690,534, filed Sep. 4, 2024, (ii) U.S. Provisional Application No. 63/701,641, filed Oct. 1, 2024, (iii) U.S. Provisional Application No. 63/701,644, filed Oct. 1, 2024, (iv) U.S. Provisional Application No. 63/690,541, filed Sep. 4, 2024, and (v) U.S. Provisional Application No. 63/701,656, filed Oct. 1, 2024, each of which is hereby incorporated by reference.

### FIELD OF THE INVENTION

This invention relates to the use of (i) a 5-HT_{2A} agonist (including 5-HT_{2A} agonists that bind to a BDNF receptor, e.g., a 5-HT_{2A} agonist that binds to a TrkB receptor or p75NTR), (ii) a therapeutic agent that promotes neuroplasticity by stimulation of the 5-HT_{2A} receptor, (iii) a therapeutic agent that releases BDNF by stimulation of the 5-HT_{2A} receptor, (iv) an alpha-amino-3-hydroxy-5-methyl-4-isoxazole propionic acid (AMPA) positive allosteric modulator, (v) a therapeutic agent that promotes neuroplasticity by stimulation of the AMPA receptor, (vi) a therapeutic agent that releases BDNF by stimulation of the AMPA receptor, or (vii) an NMDA receptor positive allosteric modulator (such as a stinel compound) in the treatment of a psychiatric condition in which depressive symptoms are prominent, including major depressive disorder (MDD), bipolar disorder, post-traumatic stress disorder, substance use disorder, and depression-related aspects of schizophrenia (e.g. negative symptoms) in select patients who, for instance, have objectively determined cognitive impairment or poor cognition (such as objectively determined impaired learning and/or memory) and/or certain EEG characteristics.

### BACKGROUND OF THE INVENTION

Clinical care for depression involves assessment and diagnosis based on a set of clinician-assessed and patient-reported symptoms such as depressed mood, anhedonia, diminished ability to think or concentrate, appetite changes, sleep and psychomotor changes but notably not based on biological or quantitative behavioral variables. When an assessment such as a magnetic resonance imaging (MRI) scan or a blood test is performed, it is to rule out non-psychiatric causes of depression which may necessitate treatments other than an antidepressant medication, including causes such as a tumor, hypothyroidism, dementia or metabolic disruptions. After diagnosing a patient with depression such as in major depressive disorder (MDD), a clinician may then prescribe one of multiple antidepressant treatments, which primarily includes drugs such as selective serotonin reuptake inhibitors (SSRIs), serotonin norepinephrine reuptake inhibitors (SNRIs), and norepinephrine dopamine reuptake inhibitors (NDRIs), or atypical antidepressants.

Notably, selection of antidepressant medication is done by trial-and-error, with no symptom profile or biological or quantitative behavioral measures to inform medication choice. Typically, SSRIs are selected as the first line treatment based on their generally better tolerability, but not because they are known to be more effective generally, nor more effective for a particular patient. Most patients, however, fail to respond adequately to the first medication, at which point selection of the next medication again follows a trial-and-error process. The next medication may be a switch (stopping one antidepressant and starting another) or adjunctive treatment (a new medication is added to the ongoing antidepressant). It has been found that on average, failing one SSRI does not necessarily predict a different response to another SSRI versus an SNRI or NDRI (29). As such, typical clinical assessments do not provide information useful for selection of subsequent medication trials, and therefore external information not available to the clinician is required for improving medication selection.

A similar situation exists for bipolar depression (including both bipolar I and bipolar II disorders). The only approved medications are atypical antipsychotic drugs, which have limited efficacy, a high rate of discontinuation due to intolerability, operate via the same mechanisms, and are selected between based on trial-and-error (41). Conventional antidepressant medications are not consistently effective and not approved by the US FDA for the treatment of bipolar depression, and carry concerns about increasing susceptibility for induction of mania.

The economic, societal and personal cost of depression is very large, with depression being the leading cause of disability worldwide. This is even more pronounced for treatment-resistant depression (28) thus suggesting that finding the best medication for an individual early in the course of treatment would provide many downstream benefits to the patient and society at large.

Similar clinical needs exist in other related conditions in which depressive symptoms exist. Such symptoms include low mood, lack of experience of pleasure (anhedonia), impairments in motivation, and impairments in attention, cognition or decision making. These conditions include major depressive disorder, bipolar depression (such as bipolar I or bipolar II disorders), post-traumatic stress disorder, substance use disorder and depression-related aspects of schizophrenia (e.g., negative symptoms). Importantly, these different depression-related symptoms or areas of dysfunction co-occur and may be functionally related. For example, a patient with major depression may report depressed mood and lack of motivation. Likewise, the same symptoms may be reported by patients diagnosed with other conditions in which similar impairments may co-occur, such as bipolar depression, post-traumatic stress disorder or substance use disorder. Though schizophrenia is often thought of with respect to prominent hallucinations and delusions, the depression-like negative symptoms are often the greater source of long-term disability and functional impairment. Hence, a treatment approach that encompasses these multiple and related functional systems would be both of importance to any one of these clinical conditions, and equally may be applicable across them.

It has been proposed that depression and depressive symptoms (e.g. in PTSD) arise at least in part from impairments in neuronal proliferation in the adult brain, neuronal growth and differentiation, elaboration of neuronal substructures important in neural communication, and impairments in neural plasticity (1-7). Moreover, it has been theorized that medications that reverse one or multiple of these dysfunctions would be effective antidepressants (1-7). It has been demonstrated, for example, that medications that have been clinically proven to be effective antidepressants in humans with depression also induce the growth and differentiation of neurons in the brains of adult animals (1, 3, 4).

There is a continuing need for improved treatments for depression, such as antidepressants which have a different mechanism of action than those currently used for treatment.

### SUMMARY OF THE INVENTION

The inventors discovered that certain patients having a psychiatric condition in which depressive symptoms are prominent, such as those patients having objectively determined poor cognition, are more receptive to treatment for their depressive symptoms with 5-HT_{2A} agonists, alpha-amino-3-hydroxy-5-methyl-4-isoxazole propionic acid (AMPA) positive allosteric modulators (AMPA PAMs), or NMDA receptor positive allosteric modulators (NMDAR PAMs) (such as stinel compounds, e.g. zelquistinel, apimostinel, or rapastinel) than similar patients who do not have poor cognition.

One embodiment is the use of one or more therapeutic agents selected from (i) a 5-HT_{2A} agonist (including 5-HT_{2A} agonists that bind to a BDNF receptor, e.g., a 5-HT_{2A} agonist that binds to the tropomyosin receptor kinase B (TrkB) receptor or low-affinity nerve growth factor receptor (p75NTR)), (ii) a therapeutic agent that promotes neuroplasticity by stimulation of the 5-HT_{2A} receptor, (iii) a therapeutic agent that releases brain-derived neurotrophic factor (BDNF) by stimulation of the 5-HT_{2A} receptor (including those therapeutic agents that stimulate the 5-HT_{2A} receptor and bind to a TrkB receptor or p75NTR), (iv) an AMPA positive allosteric modulator, (v) a therapeutic agent that promotes neuroplasticity by stimulation of the AMPA receptor, (vi) a therapeutic agent that releases BDNF by stimulation of the AMPA receptor, and (vii) an NMDA receptor positive allosteric modulator (such as a stinel compound) (therapeutic agent (i)-(vii)), to treat depression (e.g., improving depressive symptoms) in patients (i) having objectively determined cognitive impairment or poor cognition (such as objectively determined impaired learning and/or memory, e.g., impaired verbal memory, for instance as determined by VM-REACT (Verbal Memory REcAll Computerized Test)), (ii) exhibiting an electroencephalogram (EEG) with (A) a low aperiodic exponent, (B) a high power in the low gamma range (e.g., 31-50 Hz, 31-60 Hz, or 35-45 Hz), (C) a low power in the alpha frequency (8-12 Hz), or (D) any combination of any of (A), (B), and (C), or (iii) both (i) and (ii).

It was discovered that (4-benzylpiperazin-1-yl)-[2-(3-methylbutylamino)pyridin-3-yl]methanone (NSI-189) is surprisingly effective for treating depression in these select patients, as discussed in the examples provided herein. NSI-189 rapidly induces neuroplasticity, as shown by increases in long-term potentiation elicited by theta burst stimulation (42) and increases the release of BDNF (43). AMPA PAMs also lead to the release of BDNF (50-55). The inventors also discovered that multiple effects of NSI-189 on plasticity, cognition and depression-related neuronal signaling can be blocked by inhibition of the BDNF receptor TrkB. This includes release of intracellular calcium and activation of the transcription factor CREB, as well as resulting expression of downstream plasticity, depression and cognition-related genes. Without being bound by any particular theory, the inventors theorize that, since poor cognition patients having depressive symptoms were found to respond to a pro-plasticity treatment (i.e. NSI-189) and 5-HT_{2A} agonists, AMPA PAMS (56-57), and NMDAR PAMs (e.g. stinel compounds) (58-62) induce neuroplasticity, and would be effective in treating depressive symptoms in patients with poor cognition. Additionally, binding of 5-HT_{2A} agonists to TrkB has been shown to promote neuroplasticity separate of the effects on the 5-HT_{2A} receptor itself (44, 45).

One embodiment is a method of treating major depressive disorder, post-traumatic stress disorder, or one or more symptoms thereof in a human patient having (a) objectively determined cognitive impairment or poor cognition, (b) objectively determined impaired learning and/or memory, (c) an electroencephalogram (EEG) exhibiting (i) a low aperiodic exponent, (ii) a high power in the low gamma range, (iii) a low power in the alpha frequency, (iv) low power at the centro-parietal electrodes in the theta frequencies, (v) low power at the centro-parietal electrodes in the alpha frequencies, (vi) low power at the frontal electrodes in the alpha frequencies, (vii) high aperiodic exponent at one or more posterior electrodes, or (viii) any combination of any of the foregoing, or (d) any combination of any of the foregoing. The method comprises administering to the patient an effective amount of (i) a 5-HT_{2A} agonist (including 5-HT_{2A} agonists that bind to a BDNF receptor, e.g., a 5-HT_{2A} agonist that binds to the TrkB receptor or p75NTR), (ii) a therapeutic agent that promotes neuroplasticity by stimulation of the 5-HT_{2A} receptor, (iii) a therapeutic agent that releases BDNF by stimulation of the 5-HT_{2A} receptor (including those therapeutic agents that stimulate the 5-HT_{2A} receptor and bind to a TrkB receptor or p75NTR), (iv) an AMPA positive allosteric modulator, (v) a therapeutic agent that promotes neuroplasticity by stimulation of the AMPA receptor, (vi) a therapeutic agent that releases BDNF by stimulation of the AMPA receptor, and (vii) an NMDA receptor positive allosteric modulator (such as a stinel compound).

Another embodiment is a method for treating cognitive impairment, poor or slow cognition, difficulty making decisions, or reduced information processing speed as determined by an objective measurement in a human patient suffering from major depressive disorder. The method comprises administering to the patient an effective amount of a (i) a 5-HT_{2A} agonist (including 5-HT_{2A} agonists that bind to a BDNF receptor, e.g., a 5-HT_{2A} agonist that binds to the TrkB receptor or p75NTR), (ii) a therapeutic agent that promotes neuroplasticity by stimulation of the 5-HT_{2A} receptor, (iii) a therapeutic agent that releases BDNF by stimulation of the 5-HT_{2A} receptor (including those therapeutic agents that stimulate the 5-HT_{2A} receptor and bind to a TrkB receptor or p75NTR), (iv) an AMPA positive allosteric modulator, (v) a therapeutic agent that promotes neuroplasticity by stimulation of the AMPA receptor, (vi) a therapeutic agent that releases BDNF by stimulation of the AMPA receptor, and (vii) an NMDA receptor positive allosteric modulator (such as a stinel compound).

Yet another embodiment is a method of treating major depressive disorder in a human patient having reduced information processing speed, attention, memory, learning, working memory, or any combination of any of the foregoing, comprising administering to the patient an effective amount of a therapeutic agent, where the therapeutic agent is selected from (i) a 5-HT_{2A} agonist (including 5-HT_{2A} agonists that bind to a BDNF receptor, e.g., a 5-HT_{2A} agonist that binds to the TrkB receptor or p75NTR), (ii) a therapeutic agent that promotes neuroplasticity by stimulation of the 5-HT_{2A} receptor, (iii) a therapeutic agent that releases BDNF by stimulation of the 5-HT_{2A} receptor (including those therapeutic agents that stimulate the 5-HT_{2A} receptor and bind to a TrkB receptor or p75NTR), (iv) an AMPA positive allosteric modulator, (v) a therapeutic agent that promotes neuroplasticity by stimulation of the AMPA receptor, (vi) a therapeutic agent that releases BDNF by stimulation of the AMPA receptor, and (vii) an NMDA receptor positive allosteric modulator (such as a stinel compound). In one embodiment, the patient suffers from anhedonia, suicidality, or both. In another embodiment, the patient suffers from reduced information processing speed.

Yet another embodiment is a method of treating one or more symptoms selected from depressive symptoms, anhedonia, loss of interest, avolition, diminished emotional expression, inability to feel, amotivation, apathy, slow thinking, psychomotor retardation, lassitude, or any combination of any of the foregoing in a human patient suffering from post-traumatic stress disorder, bipolar depression, substance use disorder or schizophrenia, where the patient has (a) objectively determined cognitive impairment or poor cognition, (b) objectively determined impaired learning and/or memory, (c) an electroencephalogram (EEG) exhibiting (i) a low aperiodic exponent, (ii) a high power in the low gamma range, (iii) a low power in the alpha frequency, (iv) low power at the centro-parietal electrodes in the theta frequencies, (v) low power at the centro-parietal electrodes in the alpha frequencies, (vi) low power at the frontal electrodes in the alpha frequencies, (vii) high aperiodic exponent at one or more posterior electrodes, or (viii) any combination of any of the foregoing, or (d) any combination of any of the foregoing. The method comprises administering to the patient an effective amount of a therapeutic agent, where the therapeutic agent is selected from (i) a 5-HT_{2A} agonist (including 5-HT_{2A} agonists that bind to a BDNF receptor, e.g., a 5-HT_{2A} agonist that binds to the TrkB receptor or p75NTR), (ii) a therapeutic agent that promotes neuroplasticity by stimulation of the 5-HT_{2A} receptor, (iii) a therapeutic agent that releases BDNF by stimulation of the 5-HT_{2A} receptor (including those therapeutics agents that stimulate the 5-HT_{2A} receptor and bind to a TrkB receptor or p75NTR), (iv) an AMPA positive allosteric modulator, (v) a therapeutic agent that promotes neuroplasticity by stimulation of the AMPA receptor, (vi) a therapeutic agent that releases BDNF by stimulation of the AMPA receptor, and (vii) an NMDA receptor positive allosteric modulator (such as a stinel compound). In one embodiment, the patient is concurrently treated with one or more antipsychotic medications, mood stabilizers, antidepressants, or any combination of any of the foregoing.

Yet another embodiment is a method of treating negative symptoms of schizophrenia in a human patient, where the patient suffers from (a) objectively determined cognitive impairment or poor cognition, (b) objectively determined impaired learning and/or memory, (c) an electroencephalogram (EEG) exhibiting (i) a low aperiodic exponent, (ii) a high power in the low gamma range, (iii) a low power in the alpha frequency, (iv) low power at the centro-parietal electrodes in the theta frequencies, (v) low power at the centro-parietal electrodes in the alpha frequencies, (vi) low power at the frontal electrodes in the alpha frequencies, (vii) high aperiodic exponent at one or more posterior electrodes, or (viii) any combination of any of the foregoing, or (d) any combination of any of the foregoing. The method comprises administering to the patient an effective amount of a therapeutic agent, where the therapeutic agent is selected from (i) a 5-HT_{2A} agonist (including 5-HT_{2A} agonists that bind to a BDNF receptor, e.g., a 5-HT_{2A} agonist that binds to the TrkB receptor or p75NTR), (ii) a therapeutic agent that promotes neuroplasticity by stimulation of the 5-HT_{2A} receptor, (iii) a therapeutic agent that releases BDNF by stimulation of the 5-HT_{2A} receptor (including those therapeutic agents that stimulate the 5-HT_{2A} receptor and bind to a TrkB receptor or p75NTR), (iv) an AMPA positive allosteric modulator, (v) a therapeutic agent that promotes neuroplasticity by stimulation of the AMPA receptor, (vi) a therapeutic agent that releases BDNF by stimulation of the AMPA receptor, and (vii) an NMDA receptor positive allosteric modulator (such as a stinel compound). In one embodiment, the patient is concurrently treated with one or more antipsychotic medications, mood stabilizers, or any combination of any of the foregoing.

Yet another embodiment is a method of treating major depressive disorder, bipolar depression or post-traumatic stress disorder in a human patient having objectively determined impaired learning and/or memory comprising administering to the patient a therapeutic agent, where the therapeutic agent is selected from (i) a 5-HT_{2A} agonist (including 5-HT_{2A} agonists that bind to a BDNF receptor, e.g., a 5-HT_{2A} agonist that binds to the TrkB receptor or p75NTR), (ii) a therapeutic agent that promotes neuroplasticity by stimulation of the 5-HT_{2A} receptor, (iii) a therapeutic agent that releases BDNF by stimulation of the 5-HT_{2A} receptor (including those therapeutics agents that stimulate the 5-HT_{2A} receptor and bind to a TrkB receptor or p75NTR), (iv) an AMPA positive allosteric modulator, (v) a therapeutic agent that promotes neuroplasticity by stimulation of the AMPA receptor, (vi) a therapeutic agent that releases BDNF by stimulation of the AMPA receptor, and (vii) an NMDA receptor positive allosteric modulator (such as a stinel compound).

Yet another embodiment is a method of treating major depressive disorder, bipolar depression or post-traumatic stress disorder in a human patient exhibiting an electroencephalogram (EEG) with (i) a low aperiodic exponent, (ii) a high power in the low gamma range, (iii) a low power in the alpha frequency, or (iv) any combination of any of (i), (ii), and (iii) comprising administering to the patient a therapeutic agent, where the therapeutic agent is selected from (i) a 5-HT_{2A} agonist (including 5-HT_{2A} agonists that bind to a BDNF receptor, e.g., a 5-HT_{2A} agonist that binds to the TrkB receptor or p75NTR), (ii) a therapeutic agent that promotes neuroplasticity by stimulation of the 5-HT_{2A} receptor, (iii) a therapeutic agent that releases BDNF by stimulation of the 5-HT_{2A} receptor (including those therapeutic agents that stimulate the 5-HT_{2A} receptor and bind to a TrkB receptor or p75NTR), (iv) an AMPA positive allosteric modulator, (v) a therapeutic agent that promotes neuroplasticity by stimulation of the AMPA receptor, (vi) a therapeutic agent that releases BDNF by stimulation of the AMPA receptor, (vii) an NMDA receptor positive allosteric modulator (such as a stinel compound).

Yet another embodiment is a method of assessing and treating major depressive disorder in a human patient comprising:
(a) assessing whether a patient having major depressive disorder has (i) objectively determined cognitive impairment or poor cognition, (ii) objectively determined impaired learning and/or memory, (iii) an electroencephalogram (EEG) exhibiting (A) a low aperiodic exponent, (B) a high power in the low gamma range, (C) a low power in the alpha frequency, (D) low power at the centro-parietal electrodes in the theta frequencies, (E) low power at the centro-parietal electrodes in the alpha frequencies, (F) low power at the frontal electrodes in the alpha frequencies, (G) high aperiodic exponent at one or more posterior electrodes, or (H) any combination of any of the foregoing, or (iv) any combination of any of the foregoing; and
(b) upon an assessment from step (a) that the patient has (i) objectively determined cognitive impairment or poor cognition, (ii) objectively determined impaired learning and/or memory, (iii) an electroencephalogram (EEG) exhibiting (A) a low aperiodic exponent, (B) a high power in the low gamma range, (C) a low power in the alpha frequency, (D) low power at the centro-parietal electrodes in the theta frequencies, (E) low power at the centro-parietal electrodes in the alpha frequencies, (F) low power at the frontal electrodes in the alpha frequencies, (G) high aperiodic exponent at one or more posterior electrodes, or (H) any combination of any of the foregoing, or (iv) any combination of any of the foregoing, initiating administration of an effective amount of a therapeutic agent, where the therapeutic agent is selected from (i) a 5-HT_{2A} agonist (including 5-HT_{2A} agonists that bind to a BDNF receptor, e.g., a 5-HT_{2A} agonist that binds to the TrkB receptor or p75NTR), (ii) a therapeutic agent that promotes neuroplasticity by stimulation of the 5-HT_{2A} receptor, (iii) a therapeutic agent that releases BDNF by stimulation of the 5-HT_{2A} receptor (including those therapeutic agents that stimulate the 5-HT_{2A} receptor and bind to a TrkB receptor or p75NTR), (iv) an AMPA positive allosteric modulator, (v) a therapeutic agent that promotes neuroplasticity by stimulation of the AMPA receptor, (vi) a therapeutic agent that releases BDNF by stimulation of the AMPA receptor, and (vii) an NMDA receptor positive allosteric modulator (such as a stinel compound). In another embodiment, step (a) comprises objectively assessing whether the patient has impaired verbal learning and/or memory, and step (b) comprises upon an assessment from step (a) that the patient has impaired verbal learning and/or memory, initiating administration of an effective amount of the therapeutic agent. The method may further comprise step (c) upon an assessment from step (a) that the patient does not have impaired learning and/or memory, not initiating administration of the therapeutic agent.

Yet another embodiment is a method of assessing and treating major depressive disorder in a human patient comprising:
(a) assessing whether a patient having major depressive disorder has reduced information processing speed, attention, memory, learning, working memory, or any combination of any of the foregoing; and
(b) upon an assessment from step (a) that the patient has reduced information processing speed, attention, memory, learning, working memory, or any combination of any of the foregoing, initiating administration of an effective amount of a therapeutic agent, where the therapeutic agent is selected from (i) a 5-HT_{2A} agonist (including 5-HT_{2A} agonists that bind to a BDNF receptor, e.g., a 5-HT_{2A} agonist that binds to the TrkB receptor or p75NTR), (ii) a therapeutic agent that promotes neuroplasticity by stimulation of the 5-HT_{2A} receptor, (iii) a therapeutic agent that releases BDNF by stimulation of the 5-HT_{2A} receptor (including those therapeutics agents that stimulate the 5-HT_{2A} receptor and bind to a TrkB receptor or p75NTR), (iv) an AMPA positive allosteric modulator, (v) a therapeutic agent that promotes neuroplasticity by stimulation of the AMPA receptor, (vi) a therapeutic agent that releases BDNF by stimulation of the AMPA receptor, and (vii) an NMDA receptor positive allosteric modulator (such as a stinel compound).

Yet another embodiment is a method for selecting a treatment for major depressive disorder, post-traumatic stress disorder, or one or more symptoms thereof in a human patient and treating the patient comprising:
(a) diagnosing a patient as suffering from major depressive disorder, post-traumatic stress disorder, or both;
(b) objectively assessing whether the patient has impaired learning and/or memory for the purpose of selecting a treatment for the patient;
(c) selectively prescribing a therapeutic agent to the patient in view of the diagnosis of the patient and an objective assessment that the patient has impaired learning and/or memory, where the therapeutic agent is selected from (i) a 5-HT_{2A} agonist (including 5-HT_{2A} agonists that bind to a BDNF receptor, e.g., a 5-HT_{2A} agonist that binds to the TrkB receptor or p75NTR), (ii) a therapeutic agent that promotes neuroplasticity by stimulation of the 5-HT_{2A} receptor, (iii) a therapeutic agent that releases BDNF by stimulation of the 5-HT_{2A} receptor (including those therapeutic agents that stimulate the 5-HT_{2A} receptor and bind to a TrkB receptor or p75NTR), (iv) an AMPA positive allosteric modulator, (v) a therapeutic agent that promotes neuroplasticity by stimulation of the AMPA receptor, (vi) a therapeutic agent that releases BDNF by stimulation of the AMPA receptor, and (vii) an NMDA receptor positive allosteric modulator (such as a stinel compound; and
(d) upon selectively prescribing the therapeutic agent, initiating administration of the therapeutic agent to the patient. In one embodiment, step (b) comprises objectively assessing whether the patient has impaired verbal learning and/or memory, and step (c) comprises selectively prescribing the therapeutic agent to the patient in view of the diagnosis of the patient and an objective assessment that the patient has verbal impaired learning and/or memory.

Yet another embodiment is a method for determining whether a patient suffering from major depressive disorder, post-traumatic stress disorder, or both can effectively be treated with a therapeutic agent and treating a human patient receptive to such treatment, where the therapeutic agent is selected from (i) a 5-HT_{2A} agonist (including 5-HT_{2A} agonists that bind to a BDNF receptor, e.g., a 5-HT_{2A} agonist that binds to the TrkB receptor or p75NTR), (ii) a therapeutic agent that promotes neuroplasticity by stimulation of the 5-HT_{2A} receptor, (iii) a therapeutic agent that releases BDNF by stimulation of the 5-HT_{2A} receptor (including those therapeutic agents that stimulate the 5-HT_{2A} receptor and bind to a TrkB receptor or p75NTR), (iv) an AMPA positive allosteric modulator, (v) a therapeutic agent that promotes neuroplasticity by stimulation of the AMPA receptor, (vi) a therapeutic agent that releases BDNF by stimulation of the AMPA receptor, and (vii) an NMDA receptor positive allosteric modulator (such as a stinel compound). The method comprises:
(a) objectively determining whether the patient has impaired learning and/or memory; and
(b) upon a determination from step (a) that the patient has impaired learning and/or memory, initiating administration of an effective amount of (i) a 5-HT_{2A} agonist (including 5-HT_{2A} agonists that bind to a BDNF receptor, e.g., a 5-HT_{2A} agonist that binds to the TrkB receptor or p75NTR), (ii) a therapeutic agent that promotes neuroplasticity by stimulation of the 5-HT_{2A} receptor, (iii) a therapeutic agent that releases BDNF by stimulation of the 5-HT_{2A} receptor (including those therapeutic agents that stimulate the 5-HT_{2A} receptor and bind to a TrkB receptor or p75NTR), (iv) an AMPA positive allosteric modulator, (v) a therapeutic agent that promotes neuroplasticity by stimulation of the AMPA receptor, (vi) a therapeutic agent that releases BDNF by stimulation of the AMPA receptor, and (vii) an NMDA receptor positive allosteric modulator (such as a stinel compound). The method may further comprise step (c) upon a determination from step (a) that the patient does not have impaired learning and/or memory, not initiating administration to the patient of the therapeutic agent.

Yet another embodiment is a method of treating major depressive disorder, post-traumatic stress disorder, both major depressive disorder and post-traumatic stress disorder, or one or more symptoms thereof in a human patient comprising the steps of:
(a) prescribing to the patient a therapeutic agent selected from (i) a 5-HT_{2A} agonist (including 5-HT_{2A} agonists that bind to a BDNF receptor, e.g., a 5-HT_{2A} agonist that binds to the TrkB receptor or p75NTR), (ii) a therapeutic agent that promotes neuroplasticity by stimulation of the 5-HT_{2A} receptor, (iii) a therapeutic agent that releases BDNF by stimulation of the 5-HT_{2A} receptor (including those therapeutic agents that stimulate the 5-HT_{2A} receptor and bind to a TrkB receptor or p75NTR), (iv) an AMPA positive allosteric modulator, (v) a therapeutic agent that promotes neuroplasticity by stimulation of the AMPA receptor, (vi) a therapeutic agent that releases BDNF by stimulation of the AMPA receptor, and (vii) an NMDA receptor positive allosteric modulator (such as a stinel compound), the prescribing being performed in response to (i) marketing of the therapeutic agent as providing effective treatment of major depressive disorder, post-traumatic stress disorder, both major depressive disorder and post-traumatic stress disorder, or one or more symptoms thereof in patients having objectively determined impaired learning and/or memory and (ii) an objective determination that the patient has impaired learning and/or memory; and
(b) administering the prescribed therapeutic agent to the patient.

Yet another embodiment is a method of treating major depressive disorder, post-traumatic stress disorder, both major depressive disorder and post-traumatic stress disorder, or one or more symptoms thereof in a human patient comprising the steps of:
(a) diagnosing a patient as suffering from major depressive disorder, post-traumatic stress disorder, both major depressive disorder and post-traumatic stress disorder, or one or more symptoms thereof;
(b) prescribing to the patient a therapeutic agent, where the therapeutic agent is selected from (i) a 5-HT_{2A} agonist (including 5-HT_{2A} agonists that bind to a BDNF receptor, e.g., a 5-HT_{2A} agonist that binds to the TrkB receptor or p75NTR), (ii) a therapeutic agent that promotes neuroplasticity by stimulation of the 5-HT_{2A} receptor, (iii) a therapeutic agent that releases BDNF by stimulation of the 5-HT_{2A} receptor (including those therapeutic agents that stimulate the 5-HT_{2A} receptor and bind to a TrkB receptor or p75NTR), (iv) an AMPA positive allosteric modulator, (v) a therapeutic agent that promotes neuroplasticity by stimulation of the AMPA receptor, (vi) a therapeutic agent that releases BDNF by stimulation of the AMPA receptor, and (vii) an NMDA receptor positive allosteric modulator (such as a stinel compound), the prescribing being performed in response to (i) marketing of the therapeutic agent as providing effective treatment of major depressive disorder, post-traumatic stress disorder, both major depressive disorder and post-traumatic stress disorder, or one or more symptoms thereof in patients having objectively determined impaired learning and/or memory and (ii) an objective determination that the patient has impaired learning and/or memory; and
(c) administering the prescribed therapeutic agent to the patient.

Yet another embodiment is a method of treating major depressive disorder, bipolar disorder (e.g., bipolar disorder with depression), late-life depression, schizophrenia, posttraumatic stress disorder, substance use disorder, depressive symptoms or negative symptoms in a patient comprising:
(a) receiving data comprised of one or more neurophysiological measures of the patient;
(b) optionally, receiving data comprised of one or more indicators of cognitive impairment, poor or slow cognition, difficulty making decisions, reduced information processing speed, impaired learning, or impaired memory in the patient; and
(c) administering to the patient an effective amount of a therapeutic agent, where the therapeutic agent is selected from (i) a 5-HT_{2A} agonist (including 5-HT_{2A} agonists that bind to a BDNF receptor, e.g., a 5-HT_{2A} agonist that binds to the TrkB receptor or p75NTR), (ii) a therapeutic agent that promotes neuroplasticity by stimulation of the 5-HT_{2A} receptor, (iii) a therapeutic agent that releases BDNF by stimulation of the 5-HT_{2A} receptor (including those therapeutic agents that stimulate the 5-HT_{2A} receptor and bind to a TrkB receptor or p75NTR), (iv) an AMPA positive allosteric modulator, (v) a therapeutic agent that promotes neuroplasticity by stimulation of the AMPA receptor, (vi) a therapeutic agent that releases BDNF by stimulation of the AMPA receptor, and (vii) an NMDA receptor positive allosteric modulator (such as a stinel compound), where the patient is determined to be responsive to the therapeutic agent based on the data comprised of the one or more neurophysiological measures and optionally the one or more indicators of cognitive impairment, poor or slow cognition, difficulty making decisions, reduced information processing speed, impaired learning, or impaired memory. In another embodiment, the neurophysiological measure comprises electroencephalogram (EEG) recordings. In yet another embodiment, the electroencephalogram (EEG) recording of the patient exhibits low power at the centro-parietal electrodes in the theta frequencies, low power at the centro-parietal electrodes in the alpha frequencies, low power at the frontal electrodes in the alpha frequencies, high aperiodic exponent at one or more posterior electrodes, or any combination of any of the foregoing.

In one embodiment, the patient suffers from bipolar disorder, late-life depression, schizophrenia, posttraumatic stress disorder, or substance use disorder in which depressive symptoms are prominent. In another embodiment, the patient suffers from major depressive disorder. In one embodiment, the patient is concurrently treated with one or more antidepressants, antipsychotics, mood stabilizers, or any combination of any of the foregoing. In another embodiment, the patient is not concurrently treated with an antidepressant medication (other than (i) the 5-HT_{2A} agonist (including 5-HT_{2A} agonists that bind to a BDNF receptor, e.g., a 5-HT_{2A} agonist that binds to the TrkB receptor or p75NTR), (ii) the therapeutic agent that promotes neuroplasticity by stimulation of the 5-HT_{2A} receptor, (iii) the therapeutic agent that releases BDNF by stimulation of the 5-HT_{2A} receptor (including those therapeutics agents that stimulate the 5-HT_{2A} receptor and bind to a TrkB receptor or p75NTR), (iv) the AMPA positive allosteric modulator, (v) the therapeutic agent that promotes neuroplasticity by stimulation of the AMPA receptor, (vi) the therapeutic agent that releases BDNF by stimulation of the AMPA receptor, and (vii) the NMDA receptor positive allosteric modulator (such as a stinel compound)).

The neurophysiological measure can be a measure of brain activity, such as with electroencephalogram (EEG) recordings. The electroencephalogram (EEG) recordings can measure power of one or more frequencies, relative power across frequencies, power ratios between frequencies (e.g. theta-gamma power ratio), cordance, power envelope connectivity, coherence, imaginary coherence, phase locking value, phase lag index, weighted phase lag index, spatial covariance, spectrally-normalized spatial covariance, cross-frequency coupling, aperiodic exponent, alpha peak frequency, alpha peak frequency proximity, or information theoretical indices and entropies. In one embodiment, the EEG recording of the patient exhibits low power at the centro-parietal electrodes in the theta frequencies, low power at the centro-parietal electrodes in the alpha frequencies, low power at the frontal electrodes in the alpha frequencies, high aperiodic exponent at one or more posterior electrodes, or any combination of any of the foregoing.

In one embodiment, the one or more indicators of cognitive impairment, poor or slow cognition, difficulty making decisions, or reduced information processing speed comprise one or more measurements from a simple reaction time task, a choice reaction time task, a one-back working memory task, and a visual learning task, and a self-report questionnaire. In another embodiment, the one or more indicators of cognitive impairment, poor or slow cognition, difficulty making decisions, or reduced information processing speed are calculated as z-scores normalizing the patient against a healthy population. In yet another embodiment, the one or more indicators of cognitive impairment, poor or slow cognition, difficulty making decisions, or reduced information processing speed are merged into a composite cognitive task performance score.

In one embodiment, machine learning or multivariate modeling (such as described in International Publication No. WO 2020/081609 and U.S. Patent Publication Nos. 2021/0038150, 2019/126055, 2020/054888, and 2020/0401938, each of which is hereby incorporated by reference) is applied to predict the responsiveness of the patient to the administration of the therapeutic agent (i.e., (i) a 5-HT_{2A} agonist (including 5-HT_{2A} agonists that bind to a BDNF receptor, e.g., a 5-HT_{2A} agonist that binds to the TrkB receptor or p75NTR), (ii) a therapeutic agent that promotes neuroplasticity by stimulation of the 5-HT_{2A} receptor, (iii) a therapeutic agent that releases BDNF by stimulation of the 5-HT_{2A} receptor (including those therapeutic agents that stimulate the 5-HT_{2A} receptor and bind to a TrkB receptor or p75NTR), (iv) an AMPA positive allosteric modulator, (v) a therapeutic agent that promotes neuroplasticity by stimulation of the AMPA receptor, (vi) a therapeutic agent that releases BDNF by stimulation of the AMPA receptor, or (vii) an NMDA receptor positive allosteric modulator (such as a stinel compound))).

Yet another embodiment is a method of treating major depressive disorder, post-traumatic stress disorder, or one or more symptoms thereof in a human patient having objectively determined impaired learning and/or memory comprising administering to the patient an effective amount of a therapeutic agent, where the therapeutic agent is selected from (i) a 5-HT_{2A} agonist (including 5-HT_{2A} agonists that bind to a BDNF receptor, e.g., a 5-HT_{2A} agonist that binds to the TrkB receptor or p75NTR), (ii) a therapeutic agent that promotes neuroplasticity by stimulation of the 5-HT_{2A} receptor, (iii) a therapeutic agent that releases BDNF by stimulation of the 5-HT_{2A} receptor (including those therapeutic agents that stimulate the 5-HT_{2A} receptor and bind to a TrkB receptor or p75NTR), (iv) an AMPA positive allosteric modulator, (v) a therapeutic agent that promotes neuroplasticity by stimulation of the AMPA receptor, (vi) a therapeutic agent that releases BDNF by stimulation of the AMPA receptor, and (vii) an NMDA receptor positive allosteric modulator (such as a stinel compound).

In one embodiment of any of the methods described herein, prior to initiation of the administration of one of therapeutic agents (i)-(vii), no therapeutic agent (i)-(vii) was administered.

In one embodiment of any of the methods described herein, the patient has impaired learning and/or memory as objectively determined by poor immediate recall in a verbal memory test.

In one embodiment of any of the methods described herein, the patient has impaired learning and/or memory as objectively determined by poor delayed recall in a verbal memory test.

In one embodiment of any of the methods described herein, the patient is not concurrently treated with a second antidepressant medication.

In one embodiment of any of the methods described herein, the patient is concurrently treated with a second antidepressant medication.

In one embodiment of any of the methods described herein, prior to treatment with one of therapeutic agents (i)-(vii), the patient had an insufficient response to an antidepressant other than the therapeutic agents (i)-(vii).

In one embodiment of any of the methods described herein, the patient was, prior to treatment with one or more of therapeutic agents (i)-(vii), treated with one or more antidepressants and continues treatment with the one or more antidepressants during treatment with one or more of therapeutics agents (i)-(vii).

In one embodiment, the one or more antidepressants do not include a monoamine oxidase inhibitor (MAOI) or a tricyclic antidepressant.

In another embodiment, the one or more antidepressants are selected from serotonin reuptake inhibitors, serotonin and norepinephrine reuptake inhibitors, mirtazapine, bupropion, and any combination of any of the foregoing.

In yet another embodiment, the antidepressant is a standard-of-care antidepressant.

In one embodiment of any of the methods described herein, the patient exhibited an insufficient response to the antidepressant in the current depressive episode.

In one embodiment of any of the methods described herein, the patient is concurrently treated with an antipsychotic in addition to one or more therapeutic agents (i)-(vii). The antipsychotic may be quetiapine, aripiprazole, brexpiprazole, risperidone, lurasidone, iloperidone, olanzapine, ziprasidone, paliperidone, lumateperone, or cariprazine.

In one embodiment of any of the methods described herein, the human patient suffers from cognitive impairment or poor cognition as shown by one or more of a simple reaction time test, choice reaction time test, one back working memory task, and visual learning task.

In one embodiment of any of the methods described herein, the human patient suffers from cognitive impairment or poor cognition as shown by a composite score which is at least partially based upon one or more results from a simple reaction time test, choice reaction time test, one back working memory task, or visual learning task.

In one embodiment of any of the methods described herein, the patient suffers from reduced attention, memory, learning, working memory, or any combination of any of the foregoing.

In one embodiment of any of the methods described herein, the patient has objectively determined impaired verbal learning and/or memory.

In one embodiment of any of the methods described herein, the human patient suffers from impaired learning and/or memory as shown by VM-REACT (Verbal Memory REcAll Computerized Test), The Rey Auditory Verbal Learning Test (RAVLT), California Verbal Learning Test, California Verbal Learning Test—Short Form, California Verbal Learning Test—Children's Version, Hopkins Verbal Learning Test, Hopkins Verbal Learning Test—Revised, Philadelphia Verbal Learning Test, International Shopping List Test, Verbal section of the Repeatable Battery for the Assessment of Neuropsychological Status, Cerad Neuropsychological Assessment Battery Word List Task, Children's Auditory Verbal Learning Test, Children's Memory Scale, Bay Area Verbal Learning Test, Cogstate battery (which can include the following subtests: Behavioral Pattern Separation Object Test, Continuous Paired Associate Learning Test, Face Name Associative Memory Exam, Groton Maze Learning Test and its Delayed Recall and Delayed Reverse Recall versions, International Shopping List, One Card Learning Test), CANTAB (which can include the following subtests: Delayed Matching to Sample, Pattern Recognition Memory, Verbal Paired Associates, Paired Associates Learning, Verbal Recognition Memory), Penn Computerized Neurocognitive Battery (which can include the following subtests: Penn Word Memory Task, Penn Face Memory Task, Visual Object Learning Test), the NIH Toolbox and its subtests (Face Name Associative Memory Exam Test, Picture Sequence Memory Test, and Rey Auditory Verbal Learning Test), Neuropsychological Assessment Battery Memory Module, WHO/UCLA Auditory Verbal Learning Test, Repeatable Battery for the Assessment of Neuropsychological Status, Wide Range Assessment of Memory and Learning, Buschke Selective Reminding Test, Wechsler Memory Scale, Woodcock-Johnson Long Term Retrieval factor, Test of Memory and Learning, NEPSY, Brief Visuospatial Memory Test—Revised, Benton Visual Retention Test, Rey Osterreith Complex Figure Test, and any combination of any of the foregoing.

In one embodiment of any of the methods described herein, the patient suffers from major depressive disorder or one or more symptoms thereof and exhibits an electroencephalogram (EEG) with (i) a low aperiodic exponent, (ii) a high power in the low gamma range, (iii) a low power in the alpha frequency, or (iv) any combination of any of the foregoing.

In one embodiment of any of the methods for treating a major depressive episode described herein, the patient suffers from major depressive disorder.

In one embodiment of any of the methods for treating a major depressive episode described herein, the patient suffers from bipolar depression.

In one embodiment of any of the methods for treating a major depressive episode described herein, the patient suffers from bipolar I disorder.

In one embodiment of any of the methods for treating a major depressive episode described herein, the patient suffers from bipolar II disorder.

In one embodiment of any of the methods described herein, the patient suffers from anhedonia, suicidality, or both.

In one embodiment of any of the methods described herein, the patient suffers from reduced information processing speed.

In any of the methods described herein, in one embodiment, the patient is treated with one of therapeutic agents (i)-(vii) as a monotherapy.

In any of the methods described herein, in one embodiment, the patient is concurrently treated with a second antidepressant medication (e.g., an SSRI, SNRI, mirtazapine, or bupropion) in addition to the therapeutic agent (i)-(vii). In one embodiment, the patient is concurrently treated with an SSRI, SNRI, mirtazapine, or bupropion. In another embodiment, the patient is concurrently treated with an SSRI. In yet another embodiment, the patient is concurrently treated with an SNRI. In yet another embodiment, the patient is concurrently treated with mirtazapine. In yet another embodiment, the patient is concurrently treated with bupropion.

In any of the methods described herein, in one embodiment, the patient is concurrently treated with an antipsychotic in addition to the therapeutic agent (i)-(vii). In one embodiment, the antipsychotic is quetiapine, cariprazine, aripiprazole, brexpiprazole, lumateperone, or olanzapine. In another embodiment, the antipsychotic is quetiapine, aripiprazole, brexpiprazole, risperidone, lurasidone, iloperidone, olanzapine, ziprasidone, paliperidone, lumateperone, or cariprazine. In another embodiment, the antipsychotic is quetiapine, lurasidone, cariprazine, or a combination of olanzapine and fluoxetine. In yet another embodiment, the antipsychotic is a combination of brexpiprazole and sertraline. In yet another embodiment, for the treatment of major depressive disorder, the patient is concurrently treated with an antipsychotic selected from quetiapine, cariprazine, aripiprazole, brexpiprazole, and olanzapine, in addition to the therapeutic agent (i)-(vii). In yet another embodiment, for the treatment of bipolar depression, the patient is concurrently treated with an antipsychotic selected from quetiapine, lurasidone, cariprazine, lumateperone, or a combination of olanzapine and fluoxetine, in addition to the therapeutic agent (i)-(vii). In yet another embodiment, for the treatment of PTSD, the patient is concurrently treated with an antipsychotic which is a combination of brexpiprazole and sertraline, in addition to the therapeutic agent (i)-(vii).

In any of the methods described herein, in one embodiment, prior to treatment with the therapeutic agent (i)-(vii), the patient had an insufficient response to an antidepressant (e.g., an SSRI, SNRI, mirtazapine, or bupropion) other than the therapeutic agent (i)-(vii). Such a patient may be concurrently treated with the therapeutic agent (i)-(vii) and the antidepressant to which the patient had previously had an insufficient response. Alternatively, the patient may receive the therapeutic agent (i)-(vii) as a monotherapy.

In any of the methods described herein, in one embodiment, the objectively assessed impaired learning and/or memory is calculated as a standardized score (e.g., z-scores, T-scores, Standard Scores, Scaled Scores, Percentile rank, or Stanine scores) normalizing the patient against a healthy population.

In any of the methods described herein, impaired learning and/or memory may be shown by one or more of the VM-REACT (Verbal Memory REcAll Computerized Test), The Rey Auditory Verbal Learning Test, California Verbal Learning Test (including the CVLT-II and CVLT-3), California Verbal Learning Test—Short Form, California Verbal Learning Test—Children's Version, Hopkins Verbal Learning Test, Hopkins Verbal Learning Test—Revised, Philadelphia Verbal Learning Test, International Shopping List Test, Verbal section of the Repeatable Battery for the Assessment of Neuropsychological Status, Cerad Neuropsychological Assessment Battery Word List Task, Children's Auditory Verbal Learning Test, Children's Memory Scale, Bay Area Verbal Learning Test, Cogstate battery (which can include the following subtests: Behavioral Pattern Separation Object Test, Continuous Paired Associate Learning Test, Face Name Associative Memory Exam, Groton Maze Learning Test and its Delayed Recall and Delayed Reverse Recall versions, International Shopping List, One Card Learning Test), CANTAB (which can include the following subtests: Delayed Matching to Sample, Pattern Recognition Memory, Verbal Paired Associates, Paired Associates Learning, Verbal Recognition Memory), Penn Computerized Neurocognitive Battery (which can include the following subtests: Penn Word Memory Task, Penn Face Memory Task, Visual Object Learning Test), the NIH Toolbox and its subtests (Face Name Associative Memory Exam Test, Picture Sequence Memory Test, and Rey Auditory Verbal Learning Test), Neuropsychological Assessment Battery Memory Module (which can include the following subtests and their delayed recall and recognition components: List Learning, Shape Learning, Story Learning, and Daily Living Memory), WHO/UCLA Auditory Verbal Learning Test, Repeatable Battery for the Assessment of Neuropsychological Status (which includes the following subtests and their delayed recall and recognition components: List Learning, Story Memory, and Figure Recall), Wide Range Assessment of Memory and Learning (which includes the following subtests and their delayed recall and recognition components: Picture Memory, Design Learning, Story Memory, and Verbal Learning), Buschke Selective Reminding Test, Wechsler Memory Scale (which includes the following subtests and their delayed recall and recognition components: Logical Memory, Verbal Paired Associates, Designs, and Visual Reproduction), Woodcock-Johnson Long Term Retrieval factor (which includes the following subtests and their delayed recall and recognition components: Story Recall and Visual-Auditory Learning), Test of Memory and Learning (which includes the following subtests and their delayed recall and recognition components: Memory for Stories, Facial Memory, Word Selective Reminding, Visual Selective Reminding, Abstract Visual Memory, Object Recall, Visual Sequential Memory, Paired Recall, and Memory for Location), NEPSY (which includes the following subtests and their delayed recall and recognition components: List Memory, Memory for Designs, Memory for Faces, Memory for Names, Narrative Memory, Sentence Repetition, and Word List Interference), Brief Visuospatial Memory Test—Revised, Benton Visual Retention Test, Rey Osterreith Complex Figure Test, and any combination of any of the foregoing. In one preferred embodiment, impaired learning and/or memory is evaluated by the VM-REACT. In one embodiment, a composite of two or more scores regarding impaired learning and/or memory is used to access impaired learning and/or memory in a patient.

In any of the methods described herein, impaired learning and/or memory may be shown by poor immediate recall in a verbal memory test, such as the VM-REACT. In other words, the patient has impaired learning and/or memory as objectively determined by poor immediate recall in a verbal memory test, such as the VM-REACT.

In any of the methods described herein, impaired learning and/or memory may be shown by poor delayed recall in a verbal memory test, such as the VM-REACT. In other words, the patient has impaired learning and/or memory as objectively determined by poor delayed recall in a verbal memory test, such as the VM-REACT.

In one embodiment of any of the methods described herein, the patient is not concurrently treated with a second antidepressant medication.

In another embodiment of any of the methods described herein, the patient is concurrently treated with a second antidepressant medication (e.g., an SSRI, SNRI, mirtazapine, or bupropion).

In yet another embodiment of any of the methods described herein, prior to treatment with the therapeutic agent (i)-(vii), the patient had an insufficient response to an antidepressant (e.g., an SSRI, SNRI, mirtazapine, or bupropion) other than the therapeutic agent (i)-(vii) (e.g., a standard-of-care antidepressant). In one embodiment, the patient exhibited an insufficient response to the antidepressant (e.g., an SSRI, SNRI, mirtazapine, or bupropion) in the current depressive episode. In another embodiment, the patient continues treatment with the prior antidepressant concurrently with administration of the therapeutic agent (i)-(vii).

In another embodiment of any of the methods described herein, the patient is concurrently treated with an antipsychotic medication, mood stabilizer, or combination thereof in addition to the therapeutic agent (i)-(vii).

In one embodiment of any of the methods described herein, the therapeutic agent (i)-(iii) (e.g., 5-HT_{2A} agonist) is a psychedelic compound that agonizes the 5-HT_{2A} receptor, such as psilocybin, psilocin, MDMA (3,4-methylenedioxymethamphetamine), and pharmaceutically acceptable salts thereof. Other suitable 5-HT_{2A} agonists include those described in U.S. Patent Publication Nos. 2023/0219969 and 2023/0227453 and International Publication Nos. WO 2021/076572, WO 2023/114238, WO 2023/114844, WO 2023/114313, WO 2023/114325, WO 2023/114320, WO 2023/114858, WO 2023/115060, WO 2023/081753, WO 2022/221415, WO 2020/176597, WO 2020/176599, WO 2021/252691, WO 2021/252692, WO 2023/205116, WO 2024/059017, WO 2024/059090, WO 2024/035757, WO 2024/091506, WO 2022/051670, WO 2022/006186, WO 2022/235927, WO 2021/168082, WO 2022/192781, and WO 2022/261263, WO 2023/015154, and WO 2023/191952, each of which is hereby incorporated by reference.

In some embodiments, the 5-HT_{2A} agonist is selected from a lysergic acid amide, a tryptamine, a phenethylamine, and an amphetamine. In some embodiments, the 5-HT_{2A} agonist is a lysergic acid amide selected from lysergic acid diethylamide, lysergic acid 2,4-dimethylazetidine (LSZ), 6-ethyl-6-nor-lysergic acid diethylamide (ETH-LAD), 6-propyl-6-nor-Lysergic acid diethylamide (PRO-LAD), 1-Acetyl-N,N-diethyllysergamide (ALD-52), 1-propionyl-lysergic acid diethylamide (IP-LSD), NI-butyryl-lysergic acid diethylamide (1B-LSD), and NI-(cyclopropylmethanoyl)-lysergic acid diethylamide (lcP-LSD). In some embodiments, the 5-HT_{2A} agonist is selected from psilocybin, psilocin, N,N-dimethyltryptamine, 5-MeO—N,N-dimethyltryptamine, ibogaine, noribogaine, N-methyl-N-ethyltryptamine (MET), methylisopropyltryptamine (MIPT), diethyltryptamine (DET), diisopropyltryptamine (DIPT), dipropyltryptamine (DPT), ethylpropyltryptamine (EPT), 5-methoxy-methylisopropyltryptamine (5-MeO-MIPT), 5-methoxy-diisopropyltryptamine (5-MeO-DIPT), 5-methoxy-N-methyl-N-ethyltryptamine (5-MeO-MET), 5-methoxy-diethyltryptamine (5-MeO-DET), 4-hydroxy-N-methyl-N-ethyltryptamine (4-HO-MET), 4-hydroxy-methylisopropyltryptamine (4-HO-MIPT), 4-hydroxy-diisopropyltryptamine (4-HO-DIPT), 4-hydroxy-diethyltryptamine (4-HO-DET), 4-hydroxy-dipropyltryptamine (4-HO-DPT), 4-hydroxy-ethylpropyltryptamine (4-HO-EPT), 4-acetoxy-N-methyl-N-ethyltryptamine (4-AcO-MET), 4-acetoxy-methylisopropyltryptamine (4-AcO-MIPT), 4-acetoxy-diisopropyltryptamine (4-AcO-DIPT), 4-acetoxy-diethyltryptamine (4-AcO-DET), 4-acetoxy-dipropyltryptamine (4-AcO-DPT), 4-acetoxy-ethylpropyltryptamine (4-AcO-EPT), 4-acetoxy-dimethyltryptamine (4-AcO-DMT), alpha-methyltryptamine, and alpha-ethyltryptamine. In some embodiments, the 5-HT_{2A} agonist is a phenethylamine selected from mescaline, escaline, proscaline, methallylescaline, 4-bromo-2,5-dimethoxyl)enethylamine (2C-B), 2,5-dimethoxy-4-methylphenethylamine (2C-D), 2-(4-ethyl-2,5-dimethoxyphenyl)ethanamine (2C-E), 2-(2,5-dimethoxy-4-propylphenyl)ethan-1-amine (2C-P), 2-[4-(ethylsulfanyl)-2,5-dimethoxyphenyl]ethan-1-amine (2C-T-2), and 2-[2,5-dimethoxy-4-(propylsulfanyl)phenyl]ethan-1-amine (2C-T-7). In some embodiments, the 5-HT_{2A} agonist is an amphetamine selected from 2,5-dimethoxy-4-methylamphetamine (DOM), 2,5-dimethoxy-4-bromoamphetamine (DOB), 2,5-dimethoxy-4-chloroamphetamine (DOC), 2,5-dimethoxy-4-iodoamphetamine (DOI), 2,5-dimethoxy-4-ethylamphetamine (DOET), and 2,5-dimethoxy-4-propylamphetamine (DOPR). In some embodiments, the 5-HT_{2A} agonist is selected from lysergic acid diethylamide, psilocybin, psilocin, 4-AcO-DMT, N,N-dimethyltryptamine, 5-MeO—N,N-dimethyltryptamine, mescaline, 2C-B, and 2C-E. In some embodiments, the 5-HT_{2A} agonist is selected from a lysergic acid amide, ergoline, mescaline, psilocybin, bufotenin, ibogaine, psilotsin, N,N-dimethyltryptamine, and 5-MeO—N,N-dimethyltryptamine. In some embodiments, the 5-HT_{2A} agonist is lysergic acid diethylamide.

In one embodiment, the 5-HT_{2A} agonist binds to the full-length glycoprotein, known as gp145TrkB or TrkB.FL (M. Wt. 145 kDa). Examples of such 5-HT_{2A} agonists include, but are not limited to, lysergic acid diethylamide, lisuride, and psilocybin. In another embodiment, the 5-HT_{2A} agonist binds to a truncated form of TrkB lacking tyrosine kinase domain known as gp95TrkB or TrkB.T1 (M. Wt. 95 kDa).

In some embodiments, the therapeutic agent is an AMPA positive allosteric modulator. Suitable “AMPA positive allosteric modulators” (AMPA PAMs) include, but are not limited to, TAK-653 (NBI-1065845, osavampator, 9-(4-cyclohexyloxyphenyl)-7-methyl-3,4-dihydropyrazino[2,1-c][1,2,4]thiadiazine 2,2-dioxide), tulrampator (S-47445, 8-cyclopropyl-3-[2-(3-fluorophenyl)ethyl]-7H-[1,3]oxazino[6,5-g][1,2,3]benzotriazine-4,9-dione), MDI-222 ((1-[4-(1-pyrrolidinylcarbonyl)phenyl]-3-(trifluoromethyl)-4,5,6,7-tetrahydro-1H-indazole), PF-4778574 (N-[(3R,4S)-3-[4-(5-cyano-2-thienyl)phenyl]tetrahydro-2H-pyran-4-yl]-2-propanesulfonamide), pesampator (BIIB-104, PF-04958242, N-[(3S,4S)-4-[4-(5-cyanothiophen-2-yl)phenoxy]oxolan-3-yl]propane-2-sulfonamide), PF-04701475 (N-[[(5S)-3-[3-Fluoro-4-(1-pyrrolidinyl)phenyl]-4,5-dihydro-5-isoxazolyl]methyl]-2-propanesulfonamide), and GVS-111 (1-(2-phenylacetyl)-L-prolyl-glycine, ethyl ester), and pharmaceutically acceptable salts thereof. Other suitable AMPA PAMs include, but are not limited to benzamides, benzoylpyrrolidines, benzothiadiazides, biarylpropylsulfonamides, and pharmaceutically acceptable salts thereof. In some instances, the AMPA PAMs are ampakines or pharmaceutically acceptable salts thereof. In some instances, the ampakine is cyclothiazide (3-(2-bicyclo[2.2.1]hept-5-enyl)-6-chloro-1,1-dioxo-3,4-dihydro-2H-1λ6,2,4-benzothiadiazine-7-sulfonamide), LY-392098 (N-[2-[4-(2-fluorophenyl)phenyl]propyl]propane-2-sulfonamide), aniracetam (1-(4-methoxybenzoyl) pyrrolidin-2-one), CX-516 (piperidin-1-yl(quinoxalin-6-yl) methanone), CX-546 (2,3-dihydro-1,4-benzodioxin-6-yl(piperidin-1-yl) methanone), CX-614 (4,7,11-trioxa-16-azatetracyclo[8.7.0.03,8.012, 16]heptadeca-1,3(8),9-trien-17-one), CX-717 (2,1,3-Benzoxadiazol-5-yl(morpholin-4-yl) methanone), CX-1739 (N-methyl-N-(tetrahydro-2H-pyran-4-yl)benzo[c][1,2,5]oxadiazole-5-carboxamide), CX-1942, or a pharmaceutically acceptable salt thereof.

In some embodiments, the therapeutic agent is a NMDA receptor positive allosteric modulator. Suitable “NMDA receptor positive allosteric modulators” (NMDAR PAMs) include, but are not limited to, stinel compounds as well as positive allosteric modulators of GluN2A-containing NMDARs, GluN2B-containing NMDARs, GluN2C-containing NMDARs, and GluN2D-containing NMDARs. Other suitable NMDAR PAMs are disclosed in Hanson et al., Neuropsychopharmacology, 49:51-66 (2024) and Geoffroy et al., J Physiol., 2022, 600 (2): 233-259 (including Tables 1 and 2 of Geoffroy), each of which is hereby incorporated by reference, such as PYD-106, GNE-0723, GNE-5729, CIQ, (+)EU1180-453, GNE-9278, PTC-174, CAD-9303, SAGE-718, SGE-550, EU1622-14, spermine, spermidine, DMFO, PYD-106, PYD-111, GNE-3476, GNE-3419, GNE-6901, GNE-8324, UBP-551, UBP-646, UBP-753, PS, KK-169, 24-S-(HC), SGE-201, SGE-301, 83, (+)EU1180-55, (−)EU1180-55, EU1794-27, GNE-9278, and EU1622-14. Suitable stinel compounds include, but are not limited to, zelquistinel, apimostinel, rapastinel, gavestinel, licostinel, nevadistinel, and risevistinel.

In one embodiment of any of the methods described herein, the therapeutic agent (i)-(vii) is administered twice daily, once daily, once weekly, once every two weeks, twice over three weeks, twice monthly, once monthly, once every two months, or once every three months.

In one embodiment of any of the methods described herein, the therapeutic agent (i)-(vii) is orally administered.

The methods described herein can also be used to treat late-life depression in patients at least 50 years of age (e.g., at least 60 or 65 years old) (such as those with late life onset depression (first episode of depression after the age of 60)) instead of major depressive disorder.

The aforementioned methods may be used for treating one or more symptoms including depressive symptoms, anhedonia, loss of interest, avolition, diminished emotional expression, inability to feel, amotivation, apathy, slow thinking, psychomotor retardation, lassitude, or any combination of any of the foregoing, in a human patient suffering from post-traumatic stress disorder (PTSD), bipolar depression, substance use disorder, or schizophrenia instead of major depressive disorder. For instance, in one embodiment, the methods described herein are used to treat depressive symptoms of major depressive disorder, bipolar depression (such as bipolar I or bipolar II disorders), post-traumatic stress disorder, substance use disorder and depression-related aspects of schizophrenia (e.g., negative symptoms) in patients in need thereof. For example, the methods can be used to treat depressive symptoms of bipolar I depression in a patient suffering from bipolar I depression (but not major depressive disorder).

In yet another embodiment, the patient is concurrently treated with one or more antidepressants (other than the therapeutic agent (i)-(vii)) (e.g., an SSRI, SNRI, mirtazapine, or bupropion). In yet another embodiment, the patient is concurrently treated with one or more antipsychotic medications, mood stabilizers, or any combination of any of the foregoing.

The aforementioned methods may be used for treating negative symptoms of schizophrenia in a human patient instead of major depressive disorder. In another embodiment, the patient is not concurrently treated with an antidepressant medication (other than the therapeutic agent (i)-(vii)) (e.g., an SSRI, SNRI, mirtazapine, or bupropion). In yet another embodiment, the patient is concurrently treated with one or more antidepressants (other than the therapeutic agent (i)-(vii)), an antipsychotic, or mood stabilizer, or any combination of the foregoing.

The one or more indicators of cognitive impairment, slow or poor cognition or difficulty making decisions can include impaired learning and/or memory (such as impaired verbal memory). The one or more indicators of cognitive impairment, slow or poor cognition or difficulty making decisions can include, e.g., one or more measurements from a simple reaction time task, a choice reaction time task, a one-back working memory task, verbal learning and/or memory task, and a visual learning task, and a self-report questionnaire. In a preferred embodiment, the one or more indicators include measurement of learning and/or memory (e.g., VM-REACT), such as verbal memory. The indicators can also include those for learning and/or memory described herein, including the learning and/or memory tests described herein (such as VM-REACT). In one preferred embodiment, the indicator is impaired learning and/or memory as assessed by VM-REACT. Other indicators include, e.g., one or more measurements from a Mini Mental Status Exam (MMSE), Montreal Cognitive Assessment (MoCA), Repeatable Battery for the Assessment of Neuropsychological Status (RBANS), Dementia Rating Scale (DRS), Cambridge Neuropsychological Test Automated Batteries (CANTAB) and its subtests (i.e., Motor Screening, Matching to Sample Visual Search, Delayed Matching to Sample, Pattern Recognition Memory Immediate/Delayed, Spatial Recognition Memory, Paired Associate Learning, Spatial Span, Spatial Working Memory, Big Little Circle, Intra/Extradimensional Shift, Rapid Visual Processing, Reating Time, and Stockings of Cambridge), TabCAT and its subtests (i.e., dot counting, flanker, match, Running Dots, Set Shifting, Tempo, Favorites, Animal Fluency, Rapid Naming, and Quick Tap), NIH Examiner and its subtests (Dot Counting, N-Back, Flanker, Continuous Performance Test, Dysexecutive Errors, Set Shifting, Phonemic Fluency, Category Fluency, Unstructured Task, and Insight), NIH Toolbox and its subtests (Dimensional Change Card Sort Test, Face Name Associative Memory Exam Test, Flanker Inhibitory Control and Attention Test, List Sorting Working Memory Test, Oral Reading Recognition Test, Oral Symbol Digit Test, Pattern Comparison Processing Speed Test, Picture Sequence Memory Test, Picture Vocabulary Test, Rey Auditory Verbal Learning Test, Speeded Matching Test, and Visual Reasoning Test), Penn Computerized Neuropsychological Neurocognitive Battery and its subtests (Penn Conditional Exclusion Test, Penn Continuous Performance Test, Letter N-Back Task, Penn Word Memory Task, Penn Face Memory Task, Visual Object Learning Test, Penn Verbal Reasoning Test, Penn Matrix Reasoning Test, Penn Line Orientation Test, Penn Emotion Identification Test, Penn Emotion Differentiation Test, and Penn Age Differentiation Test), Cogstate battery and its subtests (Behavioral Pattern Separation Object Test, Continuous Paired Associate Learning Test, Detection Test, Face Name Associative Memory Exam, Finger Tapping Test, Groton Maze Learning Test, Identification Test, International Daily Symbol Substitution Test—Medicines, International Digit Symbol Substitution Test—Symbols, International Shopping List Test, One Back Test, One Card Learning Test, Psychomotor Vigilance Test, Social-Emotional Cognition Test, Sustained Attention Test, Sustained Attention to Response Test, and Two Back Test), Digit symbol substitution task, Oddball task, Flanker task, Wisconsin card sort task, Modified (Wisconsin) Card Sort task, Delis-Kaplan Executive Function System (D-KEFS) Sorting task, Category Test, Trail making task, D-KEFS Trail-Making Test, Spatial/Corsi Block task, Digit Span task, Backwards Digit Span task, Verbal Fluency task, D-KEFS Verbal Fluency Test, Figural/Design Fluency task, D-KEFS design fluency Test, symbol digit modalities test, Continuous Performance Test (CPT), Wechsler Adult Intelligence Scale (WAIS) coding subtest, digit vigilance test, d2 test of attention, WAIS symbol search subtest, WAIS cancellation subtest, Neuropsychological Assessment Battery (NAB) Numbers and letters subtest, NAB Digit Span subtest, Ruff 2&7 selective attention test, Stroop Color/Word test, NAB mazes and other maze tests, Spatial planning/Tower tests, Stroop test, D-KEFS Color-Word Interference Test, or Repeatable Battery for the Assessment of Neuropsychological Status (RBANS) coding subtest. In one embodiment, the indicators include one or more measurements from a typing fluency test, a verbal fluency test (such as Verbal Fluency task or D-KEFS Verbal Fluency Test), simple reaction time, choice reaction time, digit symbol substitution task, Trail-making task test (parts A and B), Wisconsin card sort task, and Flanker task. The one or more indicators of cognitive impairment can be calculated as standardized scores (e.g., z-scores, T-scores, Standard Scores, Scaled Scores, Percentile rank, Stanine scores) normalizing the patient against a healthy population. In one embodiment, the one or more indicators of cognitive impairment, slow or poor cognition (e.g., impaired memory and/or learning), and/or difficulty making decisions are merged into a composite cognitive task performance score.

In one embodiment of any of the aforementioned methods, the patient has previously been treated with one or more antidepressants (e.g., an SSRI, SNRI, mirtazapine, or bupropion) but failed to respond to them, and continues to be treated with the one or more antidepressants even after the therapeutic agent (i)-(vii) treatment is begun. In other words, the therapeutic agent (i)-(vii) is provided as an adjunctive therapy to the one or more antidepressants (e.g., an SSRI, SNRI, mirtazapine, or bupropion). In one embodiment, the one or more antidepressants do not include a monoamine oxidase inhibitor (MAOI) or a tricyclic antidepressant. In another embodiment, the one or more antidepressants are selected from serotonin reuptake inhibitors, serotonin and norepinephrine reuptake inhibitors, mirtazapine, bupropion, and any combination of any of the foregoing. The patient may suffer from major depressive disorder.

In one embodiment of any of the aforementioned methods, the patient has bipolar depression and has previously been treated with one or more mood stabilizers, antipsychotic medications, or any combination of any of the foregoing but failed to respond to them, and continues to be treated with the one or more mood stabilizers, antipsychotic medications, or any combination of any of the foregoing even after the therapeutic agent (i)-(vii) treatment is begun. In other words, the therapeutic agent (i)-(vii) is provided as an adjunctive therapy to the one or more mood stabilizers, antipsychotic medications, or any combination of any of the foregoing.

In one embodiment, the patient suffers from anhedonia. In another embodiment, the patient suffers from suicidality.

In another embodiment of any of the methods described herein, the patient suffers from both major depressive disorder and post-traumatic stress disorder.

Yet another embodiment is a system comprising:
at least one data processor; and
at least one memory storing instructions which, when executed by the at least one data processor, result in operations comprising:
(a) receiving data comprised of one or more neurophysiological measures (such as those described herein) in a patient;
(b) optionally, receiving data comprised of one or more indicators of cognitive impairment or poor cognition (such as those described herein, including impairment of learning and/or memory) in the patient;
(c) analyzing, using a multivariate model (e.g., a machine learning model), (i) the data comprised of one or more neurophysiological measures in the patient and (ii) optionally the data comprised of one or more indicators of cognitive impairment or poor cognition, to predict the responsiveness of the patient to the therapeutic agent (i)-(vii); and
(d) outputting a prediction of the responsiveness of the patient to the therapeutic agent based on the analyzed data.

Prior to step (c), the multivariate model (such as machine learning model) may be used to analyze data from prior patients receiving the therapeutic agent (i)-(vii) and one or more of their neurophysiological measures and optionally one of more of their indicators of cognitive impairment (such as impairment of learning and/or memory).

In one embodiment, the instructions result in operations further comprising outputting a recommendation to administer an effective amount of the therapeutic agent (i)-(vii).

The neurophysiological measure can be electroencephalogram (EEG) recordings, such as EEG recordings. The electroencephalogram (EEG) recordings can measure power of one or more frequencies, relative power across frequencies, power ratios between frequencies, cordance, power envelope connectivity, coherence, imaginary coherence, phase locking value, phase lag index, weighted phase lag index, spatial covariance, cross-frequency coupling, aperiodic exponent, alpha peak frequency, spectrally-normalized spatial covariance, alpha peak frequency coherence, or information theoretical indices and entropy. In one embodiment, the EEG recording of the patient exhibits low power at the centro-parietal electrodes in the theta frequencies, low power at the centro-parietal electrodes in the alpha frequencies, low power at the frontal electrodes in the alpha frequencies, high aperiodic exponent at one or more posterior electrodes, or any combination of any of the foregoing.

The one or more indicators of cognitive impairment, slow or poor cognition, impaired learning and/or memory (such as impaired verbal memory), and/or difficulty making decisions can include one or more measurements from a simple reaction time task, a choice reaction time task, a one-back working memory task, verbal memory task, and a visual learning task, and a self-report questionnaire. In a preferred embodiment, the one or more indicators include measurement of learning and/or memory (e.g., VM-REACT), such as verbal memory. The indicators can also include those for learning and/or memory described herein. Other indicators include one or more measurements from a Mini Mental Status Exam (MMSE), Montreal Cognitive Assessment (MoCA), Repeatable Battery for the Assessment of Neuropsychological Status (RBANS), Dementia Rating Scale (DRS), Cambridge Neuropsychological Test Automated Batteries (CANTAB) and its subtests (i.e., Motor Screening, Matching to Sample Visual Search, Delayed Matching to Sample, Pattern Recognition Memory Immediate/Delayed, Spatial Recognition Memory, Paired Associate Learning, Spatial Span, Spatial Working Memory, Big Little Circle, Intra/Extradimensional Shift, Rapid Visual Processing, Reating Time, and Stockings of Cambridge), TabCAT and its subtests (i.e., dot counting, flanker, match, Running Dots, Set Shifting, Tempo, Favorites, Animal Fluency, Rapid Naming, and Quick Tap), NIH Examiner and its subtests (Dot Counting, N-Back, Flanker, Continuous Performance Test, Dysexecutive Errors, Set Shifting, Phonemic Fluency, Category Fluency, Unstructured Task, and Insight), NIH Toolbox and its subtests (Dimensional Change Card Sort Test, Face Name Associative Memory Exam Test, Flanker Inhibitory Control and Attention Test, List Sorting Working Memory Test, Oral Reading Recognition Test, Oral Symbol Digit Test, Pattern Comparison Processing Speed Test, Picture Sequence Memory Test, Picture Vocabulary Test, Rey Auditory Verbal Learning Test, Speeded Matching Test, and Visual Reasoning Test), Penn Computerized Neuropsychological Neurocognitive Battery and its subtests (Penn Conditional Exclusion Test, Penn Continuous Performance Test, Letter N-Back Task, Penn Word Memory Task, Penn Face Memory Task, Visual Object Learning Test, Penn Verbal Reasoning Test, Penn Matrix Reasoning Test, Penn Line Orientation Test, Penn Emotion Identification Test, Penn Emotion Differentiation Test, and Penn Age Differentiation Test), Cogstate battery and its subtests (Behavioral Pattern Separation Object Test, Continuous Paired Associate Learning Test, Detection Test, Face Name Associative Memory Exam, Finger Tapping Test, Groton Maze Learning Test, Identification Test, International Daily Symbol Substitution Test—Medicines, International Digit Symbol Substitution Test—Symbols, International Shopping List Test, One Back Test, One Card Learning Test, Psychomotor Vigilance Test, Social-Emotional Cognition Test, Sustained Attention Test, Sustained Attention to Response Test, and Two Back Test), Digit symbol substitution task, Oddball task, Flanker task, Wisconsin card sort task, Trail making task, Corsi Block task, Digit Span task, Reverse Digit Span task, Verbal Learning and Memory task, Verbal Fluency task, symbol digit modalities test, Continuous Performance Test (CPT), Wechsler Adult Intelligence Scale (WAIS) coding subtest, digit vigilance test, d2 test of attention, WAIS symbol search subtest, WAIS cancellation subtest, Neuropsychological Assessment Battery (NAB) Numbers and letters subtest, NAB Digit Span subtest, Ruff 2&7 selective attention test, Stroop Color/Word test, NAB mazes and other maze tests, Delis-Kaplan Executive Function System (D-KEFS) design fluency subtest, or Repeatable Battery for the Assessment of Neuropsychological Status (RBANS) coding subtest. The one or more indicators of cognitive impairment, slow or poor cognition or difficulty making decisions can be calculated as standardized scores (e.g., z-scores, T-scores, Standard Scores, Scaled Scores, Percentile rank, and Stanine scores) normalizing the patient against a healthy population. These standardized scores may be calculated based on measurements such as reaction time, accuracy, memory recall accuracy, items recalled, and variation in reaction time. In one embodiment, the one or more indicators of cognitive impairment, slow or poor cognition or difficulty making decisions are merged into a composite cognitive task performance score. In one embodiment, a composite of two or more scores regarding impaired learning and/or memory is used to access impaired learning and/or memory in a patient.

In one embodiment, machine learning or multivariate modeling is applied to predict the responsiveness of the patient to the administration of the therapeutic agent (i)-(vii).

In one embodiment in any of the methods described herein, measured reduced executive function and/or attention function can be used in lieu of objectively impaired learning and/or memory. For example, one embodiment is a method of treating major depressive disorder, PTSD, bipolar depression, or one or more symptoms thereof in a human patient having measured reduced executive function and/or attention function comprising administering to the patient the therapeutic agent (i)-(vii).

### BRIEF DESCRIPTION OF THE DRAWINGS

For a more complete understanding of the present invention, including features and advantages, reference is now made to the detailed description of the invention along with the accompanying figures.

FIG. 1 has graphs showing the change in MADRS depression scores in Example 1 from baseline during stage 1 and stage 2 in (A) all patients (i.e., all-comers analysis), (B) poor cognition patients (as defined by the cognitive composite score being below the patient mean), and (C) good cognition patients (as defined by the cognitive composite score being above the patient mean) receiving placebo or 40 or 80 mg NSI-189.

FIG. 2 has graphs showing the change in MADRS depression scores in Example 1 from baseline during stage 1 and stage 2 in (A) cognitively impaired patients as defined by the cognitive composite score being below the patient mean and (B) cognitively impaired patients as defined by a CPFQ score >25 receiving placebo or 80 mg NSI-189.

FIG. 3 has graphs showing the change in MADRS scores in Example 2 from baseline in (A) poor cognition patients as defined by the cognitive composite score of z≤−0.75 relative to good cognition patients (z>−0.75), (B) poor cognition patients as defined by poor memory recall of learned word lists (verbal memory recall index) scores of z≤−0.75 relative to good cognition patients (z>−0.75), (C) subjective poor cognition patients as defined by CPFQ scores >25 relative to good cognition patients (score ≤25), and (D) subjective poor memory recall as defined by one item within the CPFQ scale (poor is moderate or greater diminishment, and good is minimal or less diminishment). Analyses control for monotherapy versus adjunctive use of NSI-189. Cohen's d's and p-values are shown on the graph.

FIG. 4 has graphs showing the change in MADRS scores in Example 2 from baseline within the group receiving NSI-189 (A) as a monotherapy, (B) as an adjunctive therapy, or (C) who had an insufficient response to at least one antidepressant and are getting NSI-189 as monotherapy or adjunctively, comparing poor cognition patients as defined by poor learning and memory (low recall index) scores of z≤−0.75 to good cognition patients (z>−0.75). Cohen's d's and p-values are shown on the graph.

FIG. 5 has graphs showing a correlation between change in MADRS scores in Example 2 from baseline to (A) week 6 or (B) week 8 and learning and memory (recall index), showing a continuous relationship between the degree of cognitive impairment (more negative z scores) and the degree of treatment response (more negative change scores). Also shown are correlation coefficients (r) and p-values for correlations between learning and memory (low recall index) and MADRS change within the NSI-189 monotherapy group, the NSI-189 adjunctive therapy group and within a group of patients who had an insufficient response to at least one antidepressant and are getting NSI-189 as monotherapy or adjunctively.

FIG. 6 has graphs showing the change in MADRS scores in Example 2 from baseline within the group receiving NSI-189 (A) as a monotherapy, (B) as an adjunctive therapy, or (C) who had an insufficient response to at least one antidepressant and are getting NSI-189 as monotherapy or adjunctively, comparing poor cognition patients as defined by learning and memory (low recall index) scores of z≤−0.418 to good cognition patients (z>−0.418). Cohen's d's and p-values are shown on the graph.

FIG. 7 shows the mean, standard error of the mean (SEM) and p-values for t-tests comparing various cognitive measures between poor cognition patients (recall index scores of z≤−0.75) to good cognition patients (scores z>−0.75) in Example 2. Poor cognition patients, even when defined by a single measure such as learning and memory, nonetheless have poorer cognition (more negative z-scores) across a wide range of cognitive tests and domains. DSST=digit-symbol substitution task, RT=reaction time, FERT=facial emotion recognition test.

FIG. 8 has graphs of (A) response rates and (B) remission rates on the MADRS (defined as a 50% or greater reduction in symptoms from baseline to week 6) in poor versus good learning and memory (recall index) patients for various definitions of the clinical population in Example 2. FIG. 8 also has a graph (C) of drug-placebo differences (plotted as Cohen's d values) for poor learning and memory patients in the second Phase 2 study and poor cognition patients in the first Phase 2 study relative to drug-placebo differences reported in all-comer depression populations receiving a range of approved antidepressants or therapeutic agents used in augmentation of or adjunctively to antidepressants (e.g., antipsychotics).

FIG. 9 has sets of graphs of EEG resting eyes closed power spectrum density plots in Example 2 comparing EEG power at different frequencies in responders (50% or greater decrease in MADRS scores from baseline) to non-responders to (A) NSI-189 (both monotherapy and adjunctive therapy), (B) who had received the drug adjunctively to an antidepressant to which they had an insufficient response, or (C) who had an insufficient response to an antidepressant in the current episode (and are getting NSI-189 as monotherapy or adjunctively to that antidepressant). Stars indicate channels with significant differences between groups (denoted as dots or lines in the upper part of each channel's panel for the corresponding frequency).

FIG. 10 has graphs showing the change in MADRS scores in Example 2 from baseline in patients receiving NSI-189 (A) as a monotherapy or adjunctive therapy, (B) adjunctively to an antidepressant to which they had an insufficient treatment response, or (C) who have had an insufficient response to an antidepressant in the current episode (regardless of whether they are receiving NSI-189 as monotherapy or adjunctively to that antidepressant), with a low aperiodic exponent (below the patient median value) versus those with a high aperiodic exponent. Analyses control for monotherapy versus adjunctive use of NSI-189. Cohen's d's and p-values are shown on the graph. The top-panel is an EEG topography plot of the channel-wise correlation between EEG resting eyes closed aperiodic exponent and percentage change from baseline in MADRS scores (a more positive correlation indicating better outcome for patients with smaller aperiodic exponents).

FIG. 11 shows the correlation between aperiodic exponent values and learning and memory performance (using recall index scores) in Example 2. The top-panel is an EEG topography plot of the channel-wise correlation between EEG resting eyes closed aperiodic exponent and recall index scores. A more positive correlation between aperiodic exponent values and learning and memory (recall index) indicates worse learning and/or memory for patients with smaller aperiodic exponents.

FIG. 12 is a graph showing the change in CAPS-5 scores in PTSD patients from Example 3 from baseline in poor cognition patients as defined by a recall index score of z≤−0.5 relative to good cognition patients (z>−0.5).

FIG. 13 is a graph of MADRS change from baseline over time in patients receiving NSI-189 with poor cognition as determined by poor verbal memory (z<−0.5) and patients with good cognition as determined by good verbal memory (z>−0.5), as described in Example 4.

FIG. 14 shows dose-response curves for intracellular calcium mobilization (panel A) and phosphorylation of transcription factor CREB (panel B) upon treatment of neural progenitor cells (NPCs) with NSI-189 or NSI-189 with TrkB antagonist ANA-12, as well as the proportion of genes that are differentially expressed by treatment with NSI-189 restored or reversed by co-treatment with ANA-12 (panel C), as described in Example 5. Asterisks indicate statistical significance, reported for the treatment factor in a two-way ANOVA for panels A and B. Mean and standard error of the mean across different experiments is reported.

FIG. 15 is a bar graph displaying the measured optical density of BDNF released into extracellular space 24 hours post treatment of NPCs with a vehicle or 0.01, 0.03, 0.1, or 0.03 μM of NSI-189.

### DETAILED DESCRIPTION OF THE INVENTION

Unless specifically stated or obvious from context, as used herein, the term “or” is understood to be inclusive.

Unless specifically stated or obvious from context, as used herein, the terms “a”, “an”, and “the” are understood to be singular or plural.

Ranges provided herein are understood to be shorthand for all of the values within the range.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein can be modified by the term about.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

Unless indicated otherwise, the term a “therapeutic agent (i)-(vii)” refers to any therapeutic agent selected from (i) a 5-HT_{2A} agonist (including 5-HT_{2A} agonists that bind to a BDNF receptor, e.g., a 5-HT_{2A} agonist that binds to the TrkB receptor or p75NTR), (ii) a therapeutic agent that promotes neuroplasticity by stimulation of the 5-HT_{2A} receptor, (iii) a therapeutic agent that releases BDNF by stimulation of the 5-HT_{2A} receptor (including those therapeutic agents that stimulate the 5-HT_{2A} receptor and bind to a TrkB receptor or p75NTR), (iv) an AMPA positive allosteric modulator, (v) a therapeutic agent that promotes neuroplasticity by stimulation of the AMPA receptor, (vi) a therapeutic agent that releases BDNF by stimulation of the AMPA receptor, and (vii) an NMDA receptor positive allosteric modulator (such as a stinel compound). Analogously, the term “therapeutic agent (i)-(iii)” refers to any therapeutic agent selected from (i) a 5-HT_{2A} agonist (including 5-HT_{2A} agonists that bind to a BDNF receptor, e.g., a 5-HT_{2A} agonist that binds to the TrkB receptor or p75NTR), (ii) a therapeutic agent that promotes neuroplasticity by stimulation of the 5-HT_{2A} receptor, and (iii) a therapeutic agent that releases BDNF by stimulation of the 5-HT_{2A} receptor (including those therapeutic agents that stimulate the 5-HT_{2A} receptor and bind to a TrkB receptor or p75NTR).

The term “monotherapy” refers to treatment with a single active agent.

Unless indicated otherwise, the term “NSI-189” refers to (4-benzylpiperazin-1-yl)-[2-(3-methylbutylamino)pyridin-3-yl]methanone, which has the structure:

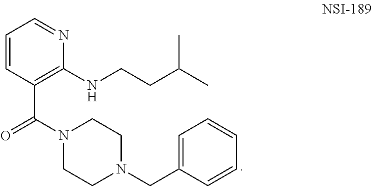 [SMILES: CC(C)CCNc1ncccc1C(=O)N1CCN(Cc2ccccc2)CC1]

The terms major depressive disorder, bipolar disorder (including bipolar I disorder and bipolar II disorder), posttraumatic stress disorder, substance use disorder and schizophrenia are intended to be as defined in the Diagnostic and Statistical Manual of Mental Disorders, 5^{th} Ed. text revision (“DSM-5-TR”), American Psychiatric Association, 2022, which is hereby incorporated by reference. The term “bipolar depression” generally refers to depressive episodes associated with bipolar I or II disorder. Bipolar disorder with depression (BD-D) is based on SCID-5 (Structured Clinical Interview for DSM-5).

As used herein, the term “brain activity” or “brain activity levels” refer to measurable (e.g., quantifiable) neural activity. Measurable neural activity includes, but is not limited to, a magnitude of activity, a frequency of activity, a delay of activity, or a duration of activity. Brain activity levels may be measured (e.g., quantified) during periods in which no stimulus is presented. In embodiments, the brain activity level measured in the absence of a stimulus is referred to as a baseline brain activity level. This may be done with eyes closed or eyes open. Alternatively, brain activity levels may be measured (e.g., quantified) when one or more stimuli are delivered (e.g., an emotional conflict task). In embodiments, the brain activity level measured in the presence of a stimulus is referred to as a brain activity level response. Brain activity levels may be measured simultaneously or sequentially throughout the whole brain, or restricted to specific brain regions (e.g., frontopolar cortex, lateral prefrontal cortex, dorsal anterior cingulate, and anterior insula). In embodiments, the brain activity level is determined relative to a baseline brain activity level taken during a baseline period. The baseline period is typically a period during which a stimulus is not presented or has not been presented for a sufficient amount of time (e.g., great than at least 0.05, 0.1, 0.15, 0.25, 0.5, 1, 2, 3, 4, 5, 10, 15, 30, 60 seconds or more).

A brain activity level may also encompass evaluating functional brain region connectivity. For example, neural activity recorded in a plurality of brain regions may have a specific time course across brain regions that can be correlated to reveal a functional brain connectivity pattern (e.g., at a first time point a first brain regions shows an increase in neural activity and at a second time point a second brain region shows an increase in activity). Thus, in embodiments, a brain activity level is a measurement (e.g., quantification) of a time course of neural activity across a plurality of brain regions. In embodiments, a brain activity level is a sequence of brain region activity levels measured (e.g., quantified) across different brain regions over time. In embodiments, a brain activity level is a functional brain region connectivity pattern.

The term “electroencephalography (EEG)” refers to a non-invasive neurophysiological technique that uses an electronic monitoring device to measure and record electrical activity in the brain. The power and aperiodic exponent may be measured across central electrodes or fronto-centro-parietal electrodes. The reference to “low power” or “high power” in a given frequency range (such as the low gamma range (e.g., 31-50 Hz, 31-60 Hz, or 35-45 Hz)) refers to a power (e.g., calculated as a standardized score, such as a z-score) below or above, respectively, that of the 50^{th} percentile, or a lower or higher cutoff, of healthy individuals of similar demographics, such as based on similarity in age to patients. The term “low aperiodic exponent” refers to an aperiodic exponent (e.g., calculated as a standardized score, such as a z-score) below that of the 50^{th} percentile, or a lower cutoff, of healthy individuals of similar demographics, such as based on similarity in age to patients. For example, the subject may have a low alpha power or a low aperiodic exponent value below the 50^{th} percentile of a similar healthy subject with a z-score less than zero, less than z=−0.25, z=−0.5, z=−0.75, z=−1, or z=−2 (e.g., with a z-score of from about −0.5 or −0.75 to about −1 or about −2, or a z-score of from about −0.75 or −1 to about −2). In one embodiment, a patient is considered to have a low alpha power or a low aperiodic exponent value when the z-score is less than −0.2, −0.25, −0.3, −0.35, −0.4, −0.45, −0.5, −0.55, −0.6, −0.65, −0.7, −0.75, −0.8, −0.85, −0.9, −0.95, or −1.0. In another embodiment, a patient is considered to have a low alpha power or a low aperiodic exponent value when the z-score is less than −1.2, −1.25, −1.3, −1.35, −1.4, −1.45, −1.5, −1.55, −1.6, −1.65, −1.7, −1.75, −1.8, −1.85, −1.9, −1.95, or −2.0. In another embodiment, the subject may have a high gamma power above the 50^{th} percentile of a similar healthy subject with a z-score greater than zero, greater than z=0.25, z=0.5, z=0.75, z=1, or z=2 (e.g., with a z-score of from about 0.5 or 0.75 to about 1 or about 2, or a z-score of from about 0.75 or 1 to about 2). In one embodiment, a patient is considered to have a high gamma power when the z-score is greater than 0.2, 0.25, 0.3, 0.35, 0.4, 0.45, 0.5, 0.55, 0.6, 0.65, 0.7, 0.75, 0.8, 0.85, 0.9, 0.95, or 1.0. In another embodiment, a patient is considered to have a high gamma power when the z-score is greater than 1.2, 1.25, 1.3, 1.35, 1.4, 1.45, 1.5, 1.55, 1.6, 1.65, 1.7, 1.75, 1.8, 1.85, 1.9, 1.95, or 2.0.

Any reference to a standardized score herein can be calculated as a z-score, T-score, Standard Score, Scaled Score, Percentile rank, or Stanine score.

The terms “treat,” “treatment,” and “treating” in the context of the administration of a therapy to a patient refers to the reduction or inhibition of the progression and/or duration of a disease or condition, the reduction or amelioration of the severity of a disease or condition, and/or the amelioration of one or more symptoms thereof resulting from the administration of one or more therapies.

Suitable 5-HT_{2A} agents used herein include (i) 5-HT_{2A} agonists, (ii) therapeutic agents that promote neuroplasticity by stimulation of the 5-HT_{2A} receptor, and (iii) therapeutic agents that release BDNF by stimulation of the 5-HT_{2A} receptor.

Suitable 5-HT_{2A} agonists that may be used in any of the methods described herein include, but are not limited to, ergolines, tryptamines, phenethylamines, LSD (lysergic acid diethylamide), psilocybin, psilocin, mescaline, 5-methoxy-N,N-dimethyltryptamine (5-MeO-DMT), N,N-dimethyltryptamine (DMT), 2,5-dimethoxy-4-iodoamphetamine (DOI), pimavanserin, 2,5-dimethoxy-4-bromoamphetamine (DOB), pharmaceutically acceptable salts thereof, and any combination of any of the foregoing.

Suitable therapeutic agents which promote neuroplasticity by stimulation of the 5-HT_{2A} receptor that may be used in any of the methods described herein include, but are not limited to, DLX-001 and DLX-007 (Delix Therapeutics) and GM-2505, G-1020, GM-5022 and GM-3009 (Gilgamesh Pharma). Additional therapeutic agents which promote neuroplasticity by stimulation of the 5-HT_{2A} receptor that may be used in any of the methods described herein include, but are not limited to, AAZ-A-154 ((R)-1-(5-methoxy-1H-indol-1-yl)-N,N-dimethylpropan-2-amine) and LED-C-233 ((R)-1-(5-fluoro-1H-indol-1-yl)-N,N-dimethylpropan-2-amine). See Dong et al., Cell, 184(1), 2779-2792, 2021, which is incorporated by reference in its entirety. Further examples of therapeutic agents which promote neuroplasticity by stimulation of the 5-HT_{2A} receptor that may be used in any of the methods described herein include are described in, for example, U.S. Pat. No. 11,344,510 and U.S. Publication Nos. 2023/0322735, 2023/0265045 and 2023/0116703, each of which is hereby incorporated by reference in its entirety.

Suitable therapeutic agents that release BDNF by stimulation of the 5-HT_{2A} receptor that may be used in any of the methods described herein include, but are not limited to, curcumin, resveratrol, ibogaine, MDMA, psilocybin, lysergic acid diethylamide, 2,5-dimethoxy-4-iodoamphetamine (DOI), and N,N-dimethyltryptamine (DMT) (46-49).

Suitable “AMPA positive allosteric modulators” (AMPA PAMs) include, but are not limited to, TAK-653 (NBI-1065845, osavampator, 9-(4-cyclohexyloxyphenyl)-7-methyl-3,4-dihydropyrazino[2,1-c][1,2,4]thiadiazine 2,2-dioxide), tulrampator (S-47445, 8-cyclopropyl-3-[2-(3-fluorophenyl)ethyl]-7H-[1,3]oxazino[6,5-g][1,2,3]benzotriazine-4,9-dione), MDI-222 ((1-[4-(1-pyrrolidinylcarbonyl)phenyl]-3-(trifluoromethyl)-4,5,6,7-tetrahydro-1H-indazole), PF-4778574 (N-[(3R,4S)-3-[4-(5-cyano-2-thienyl)phenyl]tetrahydro-2H-pyran-4-yl]-2-propanesulfonamide), pesampator (BIIB-104, PF-04958242, N-[(3S,4S)-4-[4-(5-cyanothiophen-2-yl)phenoxy]oxolan-3-yl]propane-2-sulfonamide), PF-04701475 (N-[[(5S)-3-[3-Fluoro-4-(1-pyrrolidinyl)phenyl]-4,5-dihydro-5-isoxazolyl]methyl]-2-propanesulfonamide), and GVS-111 (1-(2-phenylacetyl)-L-prolyl-glycine, ethyl ester), and pharmaceutically acceptable salts thereof. Other suitable AMPA PAMs include, but are not limited to benzamides, benzoylpyrrolidines, benzothiadiazides, biarylpropylsulfonamides, and pharmaceutically acceptable salts thereof. In some instances, the AMPA PAMs are ampakines or pharmaceutically acceptable salts thereof. In some instances, the ampakine is cyclothiazide (3-(2-bicyclo[2.2.1]hept-5-enyl)-6-chloro-1,1-dioxo-3,4-dihydro-2H-126,2,4-benzothiadiazine-7-sulfonamide), LY-392098 (N-[2-[4-(2-fluorophenyl)phenyl]propyl]propane-2-sulfonamide), aniracetam (1-(4-methoxybenzoyl) pyrrolidin-2-one), CX-516 (piperidin-1-yl(quinoxalin-6-yl) methanone), CX-546 (2,3-dihydro-1,4-benzodioxin-6-yl(piperidin-1-yl) methanone), CX-614 (4,7,11-trioxa-16-azatetracyclo[8.7.0.03,8.012, 16]heptadeca-1,3(8),9-trien-17-one), CX-717 (2,1,3-Benzoxadiazol-5-yl(morpholin-4-yl) methanone), CX-1739 (N-methyl-N-(tetrahydro-2H-pyran-4-yl)benzo[c][1,2,5]oxadiazole-5-carboxamide), CX-1942, or a pharmaceutically acceptable salt thereof.

Suitable “NMDA receptor positive allosteric modulators” (NMDAR PAMs) include, but are not limited to, stinel compounds as well as positive allosteric modulators of GluN2A-containing NMDARs, GluN2B-containing NMDARs, GluN2C-containing NMDARs, and GluN2D-containing NMDARs. Other suitable NMDAR PAMs are disclosed in Hanson et al., Neuropsychopharmacology, 49:51-66 (2024) and Geoffroy et al., J Physiol., 2022, 600 (2): 233-259 (including Tables 1 and 2 of Geoffroy), each of which is hereby incorporated by reference, such as PYD-106, GNE-0723, GNE-5729, CIQ, (+)EU1180-453, GNE-9278, PTC-174, CAD-9303, SAGE-718, SGE-550, EU1622-14, spermine, spermidine, DMFO, PYD-106, PYD-111, GNE-3476, GNE-3419, GNE-6901, GNE-8324, UBP-551, UBP-646, UBP-753, PS, KK-169, 24-S-(HC), SGE-201, SGE-301, 83, (+)EU1180-55, (−)EU1180-55, EU1794-27, GNE-9278, and EU1622-14.

The term “stinel compounds” refers to compounds with an international nonproprietary name having the suffix-stinel. Stinel compounds are a class of N-methyl-D-aspartate glutamate receptor (NMDAR) positive allosteric modulators, which typically act through a binding site of the NMDAR independent of the glycine site. Suitable stinel compounds that may be used in any of the methods described herein include, but are not limited to, zelquistinel, apimostinel, rapastinel, gavestinel, licostinel, nevadistinel, and risevistinel. See, e.g., Zhang et al., Neuropharmacology, 259, (2024) 1100100, which is hereby incorporated by reference. In one embodiment, the stinel compound is zelquistinel. In another embodiment, the stinel compound is apimostinel. In yet another embodiment, the stinel compound is rapastinel. In yet another embodiment, the stinel compound is gavestinel. In yet another embodiment, the stinel compound is licostinel. In yet another embodiment, the stinel compound is nevadistinel. In yet another embodiment, the stinel compound is risevistinel.

The term “administering” includes, but is not limited to, oral administration, administration as a suppository, topical contact, intravenous, transdermal, parenteral, intraperitoneal, intramuscular, intralesional, intrathecal, intranasal, rectal, percutaneous, or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. In embodiments, the administering does not include administration of any active agent other than the recited active agent. One preferred route of administration is the oral route.

An “effective amount” is an amount sufficient for a compound to accomplish a stated purpose relative to the absence of the compound (e.g. achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, delay, inhibition, suppression, or reduction of a symptom or symptoms of a disease or disorder, which could also be referred to as a “therapeutically effective amount.” A “reduction” of a symptom or symptoms (and grammatical equivalents of this phrase) means decreasing of the severity or frequency of the symptom(s), or elimination of the symptom(s). An “effective amount” of a drug can be an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins). Dosages may be varied depending upon the requirements of the patient and the compound being employed. The dose administered to a patient, in the context of the present disclosure, should be sufficient to affect a beneficial therapeutic response in the patient over time. The size of the dose may also be determined by the existence, nature, and extent of any adverse side-effects. Determination of the proper dosage for a particular situation is within the skill of the practitioner.

To determine efficacy of treatment in psychiatric disorders (e.g., depression, major depression, bipolar depression, or post-traumatic stress disorder (PTSD)) questionnaires (e.g., self-reporting or clinician-administered questionnaires) may be used. Non-limiting examples of questionnaires useful for assessing treatment efficacy in psychiatric disorders (e.g., depression, major depression) include the Hamilton Rating Scale for Depression (HDRS); the Hamilton Rating Scale for Depression 17 item (HDRS₁₇ or HDRS-17); the 21 item HDRS (HDRS₂₁); the 24 item HDRS (HDRS₂₄); the Quick Inventory of Depressive Symptoms (QIDS); the Patient Health Questionnaire (PHQ-9); the Cognitive and Physical Functioning Questionnaire (CPFQ); the Mood and Symptom Questionnaire subscale scores for Anxious Arousal, Anhedonic Depression, and General Distress; the Montgomery-Asberg Depression Scale (MADRS); the Beck Depression Inventory; the Clinical Global Impressions (CGI) scale); and the Snaith-Hamilton Pleasure Scale (SHAPS). Questionnaires may be completed prior to, during, and following treatment, and changes in the scores may be used to determine treatment efficacy. In some embodiments, the HDRS 17 is used to determine treatment efficacy. In some embodiments, the HDRS is used to determine treatment efficacy. In some embodiments, the HDRS₂₁ is used to determine treatment efficacy. In embodiments, the HDRS₂₄ is used to determine treatment efficacy. In some embodiments, the QIDS is used to determine treatment efficacy. In some embodiments, the Mood and Symptom Questionnaire subscale scores for Anxious Arousal, Anhedonic Depression, and General Distress are used to determine treatment efficacy. In some embodiments, the MADRS is used to determine treatment efficacy. In embodiments, the Beck Depression Inventory is used to determine treatment efficacy. In some embodiments, the clinical global impression (CGI) scale is used to determine treatment efficacy. In some embodiments, treatment efficacy is determined by measuring (e.g., quantifying) a change in the HDRS₆, MADRS₆, or HDRS₁₇ score. In some embodiments, treatment efficacy is determined by measuring (e.g., quantifying) a change in the HDRS₂₁ score. In some embodiments, treatment efficacy is determined by measuring (e.g., quantifying) a change in the HDRS score. In some embodiments, treatment efficacy is determined by measuring (e.g., quantifying) a change in the HDRS₂₄ score. In some embodiments, treatment efficacy is determined by measuring (e.g., quantifying) a change in the QIDS score. In some embodiments, treatment efficacy is determined by measuring (e.g., quantifying) a change in the Mood and Symptom Questionnaire subscale scores for Anxious Arousal, Anhedonic Depression, and General Distress. In some embodiments, treatment efficacy is determined by measuring (e.g., quantifying) a change in the MADRS score. In embodiments, treatment efficacy is determined by measuring (e.g., quantifying) a change in the Beck Depression Inventory score. In some embodiments, treatment efficacy is determined by measuring (e.g., quantifying) a change in the CGI scale. A non-limiting example of a questionnaire useful for assessing treatment efficacy for PTSD is the Clinician-Administered PTSD Scale for DSM-5 (CAPS-5). In another embodiment, the questionnaire for assessing treatment efficacy of PTSD is the Clinician-Administered PTSD Scale for DSM-IV (CAPS-IV). In yet another embodiment, the questionnaire for assessing treatment efficacy of PTSD is a self-report, such as the PCL (PTSD Checklist) (e.g., the PTSD Checklist for DSM-5 (PCL-5)). In some embodiments, treatment efficacy is determined by measuring (e.g., quantifying) a score on a questionnaire as described herein during a baseline period prior to treatment to a score on a questionnaire as described herein reported 1, 2, 3, 4, 6, 8 or more weeks after commencing treatment or terminating treatment.

Treatment may result in a reduction of symptoms (e.g., a response) or in remission. In embodiments, a reduction in symptoms is referred to as a response. In embodiments, a response is a 50% or greater decrease in symptoms. A response (e.g., a 50% or greater decrease in symptoms) to treatment may be determined by measuring (e.g., quantifying) a change in a score as described herein, including embodiments thereof, on a questionnaire as described herein, including embodiments thereof. In embodiments, remission is a score of 7 or less at endpoint on the HDRS₁₇. In embodiments, remission is a score of 7 or less at endpoint on the HDRS. In embodiments, remission is a score of 10 or less on the HDRS₂₄. In embodiments, remission is a score of 5 or less on the QIDS. In embodiments, remission is a score of 10 or 9 or less on the MADRS. In embodiments, remission is a score of 4 or less on the PHQ9.

“Learning and/or memory” can be objectively assessed by one or more of the tests described herein, including the following tests: VM-REACT (Verbal memory recall computerized test), The Rey Auditory Verbal Learning Test, California Verbal Learning Test (including the CVLT-II and CVLT-3), California Verbal Learning Test—Short Form, California Verbal Learning Test—Children's Version, Hopkins Verbal Learning Test, Hopkins Verbal Learning Test—Revised, Philadelphia Verbal Learning Test, International Shopping List Test, Verbal section of the Repeatable Battery for the Assessment of Neuropsychological Status, Cerad Neuropsychological Assessment Battery Word List Task, Children's Auditory Verbal Learning Test, Children's Memory Scale, Bay Area Verbal Learning Test, Cogstate battery (which can include the following subtests: Behavioral Pattern Separation Object Test, Continuous Paired Associate Learning Test, Face Name Associative Memory Exam, Groton Maze Learning Test and its Delayed Recall and Delayed Reverse Recall versions, International Shopping List, One Card Learning Test), CANTAB (which can include the following subtests: Delayed Matching to Sample, Pattern Recognition Memory, Verbal Paired Associates, Paired Associates Learning, Verbal Recognition Memory), Penn Computerized Neurocognitive Battery (which can include the following subtests: Penn Word Memory Task, Penn Face Memory Task, Visual Object Learning Test), the NIH Toolbox and its subtests (Face Name Associative Memory Exam Test, Picture Sequence Memory Test, and Rey Auditory Verbal Learning Test), Neuropsychological Assessment Battery Memory Module (which can include the following subtests and their delayed recall and recognition components: List Learning, Shape Learning, Story Learning, and Daily Living Memory), WHO/UCLA Auditory Verbal Learning Test, Repeatable Battery for the Assessment of Neuropsychological Status (which includes the following subtests and their delayed recall and recognition components: List Learning, Story Memory, and Figure Recall), Wide Range Assessment of Memory and Learning (which includes the following subtests and their delayed recall and recognition components: Picture Memory, Design Learning, Story Memory, and Verbal Learning), Buschke Selective Reminding Test, Wechsler Memory Scale (which includes the following subtests and their delayed recall and recognition components: Logical Memory, Verbal Paired Associates, Designs, and Visual Reproduction), Woodcock-Johnson Long Term Retrieval factor (which includes the following subtests and their delayed recall and recognition components: Story Recall and Visual-Auditory Learning), Test of Memory and Learning (which includes the following subtests and their delayed recall and recognition components: Memory for Stories, Facial Memory, Word Selective Reminding, Visual Selective Reminding, Abstract Visual Memory, Object Recall, Visual Sequential Memory, Paired Recall, and Memory for Location), NEPSY (which includes the following subtests and their delayed recall and recognition components: List Memory, Memory for Designs, Memory for Faces, Memory for Names, Narrative Memory, Sentence Repetition, and Word List Interference), Brief Visuospatial Memory Test—Revised, Benton Visual Retention Test, Rey Osterreith Complex Figure Test, and any combination of any of the foregoing. The VM-REACT (Verbal Memory REcAll Computerized Test) is described in Naparstek et al., J Psychiatr Res., 2019, 114:170-177 (PMID 31096177), which is hereby incorporated by reference. In one embodiment, the learning and/or memory ability of a patient is objectively assessed by the VM-REACT (Verbal memory recall computerized test).

Memory is often assessed by a recall index. The term “recall index” refers to a measure of accuracy of immediate and/or delayed recall of the learned material. For example, immediately after learning, recall can be assessed, and again after a delay (such as 15-30 minutes, e.g., 20 minutes). Alternatively, only one of these conditions may be used to assess recall. Similarly, an irrelevant distractor learning trial may be given to further separate the learning whose memory is being assessed from the assessment of the memory per se. The recall index therefore encompasses one or more assessments of recall after learning has occurred.

The term “impaired learning and/or memory” refers to a subject having learning and/or memory, as measured by one or more tests, below that of the 50^{th} percentile, or a lower cutoff, of healthy subjects of similar demographics, such as based on similarity in age to patients (i.e., z-score <0). The z-score is an example of a standardized score that can be used to characterize subjects as “impaired”. The z-score reflects a transformation of learning and/or memory performance relative to a healthy subject distribution, which may account for factors such as age, education and gender in that transformation. A z score below zero indicates performance for that subject that is below the 50^{th} percentile of similar healthy subjects, while a z score above zero indicates performance that is above the 50^{th} percentile of similar healthy subjects. For example, the subject may have a learning and/or memory score below the 50^{th} percentile of a similar healthy subject with a z-score less than zero, less than z=−0.25, z=−0.5, z=−0.75, z=−1, or z=−2 (e.g., with a z-score of from about −0.5 or −0.75 to about −1 or about −2, or a z-score of from about −0.75 or −1 to about −2). In one embodiment, a patient is considered to have impaired learning and/or memory when the z-score is less than −0.2, −0.25, −0.3, −0.35, −0.4, −0.45, −0.5, −0.55, −0.6, −0.65, −0.7, −0.75, −0.8, −0.85, −0.9, −0.95, or −1.0. In another embodiment, a patient is considered to have impaired learning and/or memory when the z-score is less than −1.2, −1.25, −1.3, −1.35, −1.4, −1.45, −1.5, −1.55, −1.6, −1.65, −1.7, −1.75, −1.8, −1.85, −1.9, −1.95, or −2.0. In one embodiment, impaired learning and/or memory is assessed (partly or wholly) based on recall index.

In one embodiment, a patient (such as one having major depressive disorder, a major depressive episode, bipolar depression, or post-traumatic stress disorder) is considered to have impaired learning and/or memory when the z-score as determined by VM-REACT is less than −0.5. In another embodiment, a patient (such as one having major depressive disorder, a major depressive episode, bipolar depression, or post-traumatic stress disorder) is considered to have impaired learning and/or memory when the z-score as determined by VM-REACT is less than −0.30. In yet another embodiment, a patient (such as one having major depressive disorder, a major depressive episode, bipolar depression, or post-traumatic stress disorder) is considered to have impaired learning and/or memory when the z-score as determined by VM-REACT is less than −0.35. In yet another embodiment, a patient (such as one having major depressive disorder, a major depressive episode, bipolar depression, or post-traumatic stress disorder) is considered to have impaired learning and/or memory when the z-score as determined by VM-REACT is less than −0.40. In yet another embodiment, a patient (such as one having major depressive disorder, a major depressive episode, bipolar depression, or post-traumatic stress disorder) is considered to have impaired learning and/or memory when the z-score as determined by VM-REACT is less than −0.45. In yet another embodiment, a patient (such as one having major depressive disorder, a major depressive episode, bipolar depression, or post-traumatic stress disorder) is considered to have impaired learning and/or memory when the z-score as determined by VM-REACT is less than −0.55. In yet another embodiment, a patient (such as one having major depressive disorder, a major depressive episode, bipolar depression, or post-traumatic stress disorder) is considered to have impaired learning and/or memory when the z-score as determined by VM-REACT is less than −0.60. In yet another embodiment, a patient (such as one having major depressive disorder, a major depressive episode, bipolar depression, or post-traumatic stress disorder) is considered to have impaired learning and/or memory when the z-score as determined by VM-REACT is less than −0.65. In yet another embodiment, a patient (such as one having major depressive disorder, a major depressive episode, bipolar depression, or post-traumatic stress disorder) is considered to have impaired learning and/or memory when the z-score as determined by VM-REACT is less than −0.70. In yet another embodiment, a patient (such as one having major depressive disorder, a major depressive episode, bipolar depression, or post-traumatic stress disorder) is considered to have impaired learning and/or memory when the z-score as determined by VM-REACT is less than −0.75. In yet another embodiment, a patient (such as one having major depressive disorder, a major depressive episode, bipolar depression, or post-traumatic stress disorder) is considered to have impaired learning and/or memory when the z-score as determined by VM-REACT is less than −1.0.

The term “poor cognition” (or “cognitively poor”), unless otherwise defined, refers to a subject having cognitive function, as measured by one or more tests of cognitive function, below that of the 50^{th} percentile of healthy subjects of similar demographics, such as age (z-score <0). Z scores reflect a transformation of cognitive task performance relative to a healthy subject distribution, which may account for factors such as age, education and gender in that transformation. A z score below zero indicates performance for that subject that is below the 50^{th} percentile of similar healthy subjects, while a z score above zero indicates performance that is above the 50^{th} percentile of similar healthy subjects. For example, the subject may have a cognitive score below the 50^{th} percentile of a similar healthy subject with a z-score less than zero, less than z=−0.25, z=−0.30, z=−0.35, z=−0.40, z=−0.45, z=−0.5, z=−0.75, z=−1, or z=−2 (e.g., with a z-score of from about −0.5 or −0.75 to about −1 or about −2, or a z-score of from about −0.75 or −1 to about −2).

Cognition can be assessed by methods known in the art, including those described in DSM-5 (see, e.g., pages 593-595). For instance, cognition can be measured by a simple reaction time test, choice reaction time test, one back working memory task, visual learning task, learning and/or memory (such as verbal learning and/or memory), or any combination of any of the foregoing. In one embodiment, learning and/or memory is assessed. In one embodiment, the cognitive ability of a subject is measured with a Cogstate Brief Battery as described in Maruff et al., Arch Clin Neuropsychol. 2009, 24(2):165-78, which is hereby incorporated by reference. Tests of cognition (such as to assess information processing speed, working memory, learning, and attention) include, but are not limited to, Digit symbol substitution task, Oddball task, Flanker task, Wisconsin card sort task, Trail making task, Corsi Block task, Digit Span task, Reverse Digit Span task, Verbal Learning and/or memory task, and Verbal Fluency task. Reduced information processing speed, slow decision making or difficulty making decisions may be diagnosed by tests that assess reaction times or performance under speed-based task instructions (e.g., reduced number of correct symbols in a digit symbol substitution task or reduced verbal fluency in a fixed amount of allotted time), such as those described in J. DeLuca and J. H. Kalmar, Information Processing Speed in Clinical Populations, Taylor & Francis Group (2008), which is hereby incorporated by reference. Decision making (including slow decision making and difficulty making decisions) can be assessed by the performance of tasks that assess the process of deciding in the face of competing alternatives (e.g., simulated gambling) (DSM-5, p. 593). Reductions in attention can be assessed by: (1) for sustained attention: maintenance of attention over time (e.g., pressing a button every time a tone is heard, and over a period of time), (2) for selective attention: maintenance of attention despite competing stimuli and/or distractors: hearing numbers and letters read and asked to count only letters, and (3) for divided attention: attending to two tasks within the same time period: rapidly tapping while learning a story being read. Processing speed can be quantified on any task by timing it (e.g., time to put together a design of blocks; time to match symbols with numbers; speed in responding, such as counting speed or serial 3 speed).

Reductions in working memory can be assessed by the ability to hold information for a brief period and to manipulate it (e.g., adding up a list of numbers, repeating a series of numbers or words backward or repeating a sequence of actions).

Reductions in memory can also be assessed by the following methods (in addition to the working memory assessment method described above):

(1) Immediate memory span: Ability to repeat a list of words, digits or sequence of actions.

(2) Learning and retention: Assesses the process of encoding new information (e.g., word lists (with the word list having the potential to also be repeated multiple times), a short story, or diagrams). Reduced acquisition and retention of new information may be diagnosed by tests that assess learning and memory (e.g., learning a list of words or symbols over a series of trials and recalling them following a 15- to 30-minute delay; learning a brief story or complex figure and recalling them following a 20- to 30-minute delay), such as those described in E. Strauss, E. M. S. Sherman, and O. Spreen (2006), A compendium of neuropsychological tests: Administration, norms, and commentary. (3rd Edition). Oxford University Press, New York, New York, which is hereby incorporated by reference. The aspects of recent memory that can be tested include 1) free recall (e.g. the person is asked to recall as many words, diagrams, or elements of a story as possible); 2) cued recall (e.g. semantic cues such as “List all the food items on the list” or “Name all of the children from the story” are provided to the subject); 3) recognition memory (e.g., “Was ‘apple’ on the list?” or “Did you see this diagram or figure?”); and 4) recall of an original list of items or words after presentation of a distractor list of items or words. It can also be assessed as a combination of different aspects of recent memory; for example, recall of an original list of items or words following a distractor list and free recall, such as those described in R. J. Ivnik, J. F. Malec, E. G. Tangalos, R. C. Petersen, E. Kokmen, and L. T. Kurland (1992), Mayo's Older Americans Normative Studies: Updated AVLT norms for ages 56 to 97, The Clinical Neuropsychologist 6, 83-104. Other aspects of memory that can be assessed include semantic memory (memory for facts), autobiographical and episodic memory (memory for personal events or people), and implicit (procedural) learning (unconscious learning of skills).

The term “slow cognition”, unless otherwise defined, refers to a subject having slow cognitive function (longer time to respond), as measured by one or more tests of information processing speed (such as a simple reaction time test or choice reaction time test), below the 50^{th} percentile, or another cutoff, of a healthy subject of similar age (e.g., z-score <0).

Processing speed can be quantified on any task by timing it (e.g., time to put together a design of blocks; time to match symbols with numbers; speed in responding, such as counting speed or serial 3 speed).

Reduced learning and/or memory can be assessed by the methods described above for immediate memory span and learning and retention.

Reduced executive function can be assessed by tests that evaluate flexibility of thinking (e.g., alternating between numbers and letters in sequential order), abstract/conceptual reasoning and problem-solving (e.g., completing complex puzzles), planning (e.g., completing mazes), organization (e.g., categorizing a list of words based on semantic cues), working memory (e.g., holding and manipulating information held in one's mind), creativity, generativity, and initiation (e.g., spontaneously producing words that begin with a specific letter), impulse control and inhibition (e.g., purposefully suppressing automatic responses to test stimuli), and self-monitoring (e.g., checking answers to ensure accuracy), such as those described in E. Strauss, E. M. S. Sherman, and O. Spreen (2006), G. A. Gioia, P. K. Isquith, S. C. Guy, and L. Kenworthy (2015), Behavior Rating Inventory of Executive Function®, Second Edition (BRIEF® 2). Lutz, FL: PAR Inc.; and Delis, D. C., Kaplan, E., & Kramer, J. H. (2001), Delis-Kaplan Executive Function System (D-KEFS), The Psychological Corporation, San Antonio, TX. Gioia G. A., Isquith P. K., Guy S. C., Kenworthy L. (2015).

In one embodiment, the cognitive impairment, poor or slow cognition or difficulty making decisions is due, at least in part, to reduced attention, memory, learning, working memory, or any combination of any of the foregoing.

As used herein, the terms “subject,” “participant,” and “patient” are used interchangeably and refer to a human patient unless indicated otherwise.

Suitable antidepressants for concurrent therapy include, but are not limited to, (i) serotonin and norepinephrine reuptake inhibitors (SNRIs) (such as venlafaxine, duloxetine, milnacipran, sibutramine, SEP-227162, or LY 2216684), (ii) selective serotonin reuptake inhibitors (SSRIs) (such as escitalopram, fluoxetine, fluvoxamine, sertraline, citalopram, vilazodone, and paroxetine), (iii) atypical antidepressants (such as agomelatine, mianserin, mirtazapine, nefazodone, opipramol, tianeptine, and trazodone), and (iv) norepinephrine and dopamine reuptake inhibitors (NDRIs) (such as bupropion, amineptine, prolintane, dexmethylphenidate, and pipradrol). In one embodiment, the antidepressant is selected from SSRIs, SNRIs, mirtazapine, bupropion, or any combination of any of the foregoing. In another embodiment, the antidepressant is not a monoamine oxidase inhibitor (MAOI), a tricyclic antidepressant (TCA) (such as amitriptyline, imipramine, clomipramine, and desipramine), or ketamine.

Suitable mood stabilizers include, but are not limited to, lithium carbonate, divalproex sodium, valproic acid, valproate semisodium, sodium valproate, tiagabine, levetiracetam, lamotrigine, gabapentin, carbamazepine, oxcarbazepine, topiramate, zonisamide, aripiprazole, risperidone, olanzapine, quetiapine, asenapine, paliperidone, ziprasidone, lurasidone, lumateperone, cariprazine, verapamil, clonidine, propranolol, mexiletine, guanfacine and omega-3 fatty acids.

In certain embodiments of any of the methods described herein, the impairment in learning and/or memory is measured using one or more of the following learning and/or memory tests:

VM-REACT
Rey Auditory Verbal Learning Test
California Verbal Learning Test (including the CVLT-II and CVLT-3)
California Verbal Learning Test - Short Form
California Verbal Learning Test - Children's Version
Hopkins Verbal Learning Test - Revised
Neuropsychological Assessment Battery Memory Module, which includes
the following subtests and their delayed recall and recognition
components:
List Learning
Shape Learning
Story Learning
Daily Living Memory
WHO/UCLA Auditory Verbal Learning Test
Philadelphia Verbal Learning Test
Cerad Neuropsychological Assessment Battery Word List Task
Children's Auditory Verbal Learning Test
Children's Memory Scale
Bay Area Verbal Learning Test
International Shopping List Task
Cogstate Battery, which includes the following subtests and their
delayed recall and recognition components:
Behavioral Pattern Separation Object Test
Continuous Paired Associate Learning Test
Face Name Associative Memory Exam
Groton Maze Learning Test
International Shopping List
One Card Learning Test
CANTAB battery, which includes the following subtests and their
delayed recall and recognition components:
Delayed Matching to Sample
Pattern Recognition Memory
Verbal Paired Associates
Paired Associates Learning
Verbal Recognition Memory
Penn Computerized Neurocognitive Battery, which includes the
following subtests and their delayed recall and recognition
components:
Penn Word Memory Task
Penn Face Memory Task
Visual Object Learning Test
NIH Toolbox, which includes the following subtests and their
delayed recall and recognition components:
Face Name Associative Memory Exam Test
Picture Sequence Memory Test
Rey Auditory Verbal Learning Test
Repeatable Battery for the Assessment of Neuropsychological Status,
which includes the following subtests and their delayed recall and
recognition components:
List Learning
Story Memory
Figure Recall
Wide Range Assessment of Memory and Learning, which includes the
following subtests and their delayed recall and recognition
components:
Picture Memory
Design Learning
Story Memory
Verbal Learning
Buschke Selective Reminding Test
Wechsler Memory Scale, which includes the following subtests and
their delayed recall and recognition components:
Logical Memory
Verbal Paired Associates
Designs
Visual Reproduction
Woodcock-Johnson Long Term Retrieval factor, which includes the
following subtests and their delayed recall and recognition
components:
Story Recall
Visual-Auditory Learning
Test of Memory and Learning, which includes the following subtests
and their delayed recall and recognition components:
Memory for Stories
Facial Memory
Word Selective Reminding
Visual Selective Reminding
Abstract Visual Memory
Object Recall
Visual Sequential Memory
Paired Recall
Memory for Location
NEPSY, which includes the following subtests and their delayed
recall and recognition components:
List Memory
Memory for Designs
Memory for Faces
Memory for Names
Narrative Memory
Sentence Repetition
Word List Interference
Brief Visuospatial Memory Test - Revised
Benton Visual Retention Test
Rey Osterreith Complex Figure Test

The term “advertising” refers to notifying, informing, and/or apprising one or more individuals of information (e.g., the efficacy of a pharmaceutical product containing the therapeutic agent (i)-(vii) (as described herein) in the treatment of major depressive disorder, post-traumatic stress disorder, or one or more symptoms thereof in patients having impaired learning and/or memory), such as by mass media, including, but not limited to, newspaper, magazine, and internet advertisements, television commercials, and billboard signs. The term “advertising” as used herein also includes including a statement that a pharmaceutical product containing the therapeutic agent (i)-(vii) (as described herein) can treat major depressive disorder, post-traumatic stress disorder, or one or more symptoms thereof in patients having impaired learning and/or memory in the labeling for the pharmaceutical product.

The term “marketing” refers to the act or process of selling a product (e.g., a pharmaceutical product containing the therapeutic agent (i)-(vii) (as described herein)), including, but not limited to, any offer for sale or sale of a product, as well as advertising. The marketing may be directed to, for example, doctors (such as psychiatrists or general practitioners) treating human subjects suffering from major depressive disorder, post-traumatic stress disorder, or one or more symptoms thereof. The marketing step may comprise the step of including a statement in the labelling for a pharmaceutical product containing the therapeutic agent (i)-(vii) (as described herein) can effectively treat major depressive disorder, post-traumatic stress disorder, or one or more symptoms thereof in a patient having impaired learning and/or memory.

In any of the methods described herein, the therapeutic agent (i)-(vii) (as described herein) may be incorporated into a pharmaceutical product. The pharmaceutical product may be a therapeutic package which comprises (a) one or more dosage forms (e.g., tablets) of the therapeutic agent (i)-(vii) (as described herein), (b) a container that contains one or more of the dosage forms, and (c) written matter such as labelling directing the use of the dosage forms in the treatment of major depressive disorder, post-traumatic stress disorder, or one or more symptoms thereof in patients having impaired learning and/or memory.

The term “pharmaceutical product” refers to any pharmaceutical product containing the therapeutic agent (i)-(vii) (e.g., a 5-HT_{2A} agent) (as described herein). The pharmaceutical product may contain one or dosage forms (e.g., tablets) of the therapeutic agent (i)-(vii) (e.g., a 5-HT_{2A} agent) (as described herein).

### Example 1—Phase 2 Clinical Trial with NSI-189

A retrospective analysis of the 220-patient phase 2 study of NSI-189 discussed above was performed (11). In the study, patients having major depressive disorder were randomized to receive a total daily dose of 40 mg or 80 mg of NSI-189 or placebo in stage 1 (first 6 weeks) of the clinical trial design. Patients receiving placebo and who failed to respond to placebo were then re-randomized to 40 mg, 80 mg or placebo in stage 2 (second 6 weeks) of the trial. The remaining patients continued their treatment for stage 2 of the trial. Treatment in either stage was given for six weeks. Patients were eligible for study participation if they were between the ages of 18-60 years, with current major depressive disorder of at least 8 weeks duration according to the DSM-5, as diagnosed by the Structured Clinical Interview for the DSM-5 clinical trial version (SCID-5-CT) during the screen and remote assessment visits, and if they were scored at least 20 at screen, remote assessment, and baseline visits on the Montgomery-Asberg Depression Rating Scale (MADRS). The phase 2 study collected cognitive task performance data prior to treatment, and then again after both stage 1 and stage 2 of the treatment protocol. The purpose of doing so was to determine whether treatment with this compound results in improvement in cognitive functioning, as measured by a variety of behavioral measures.

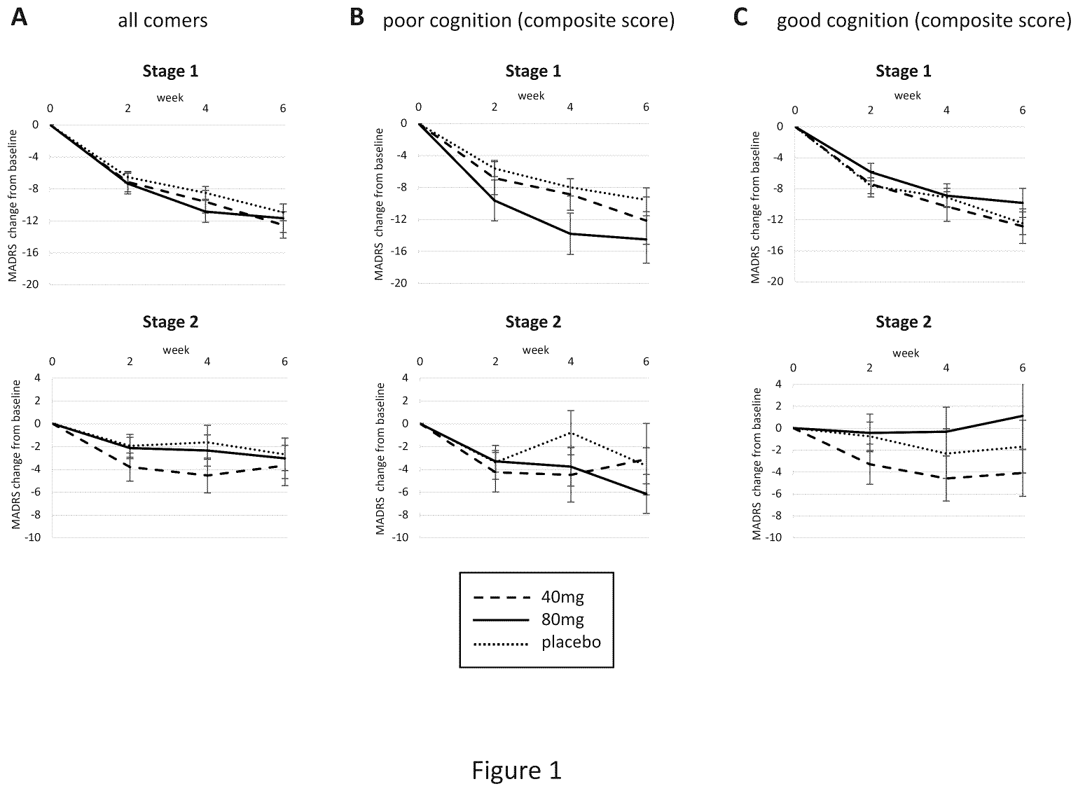

Prior to this retrospective analysis, there was no consideration as to using behavioral measures to predict treatment outcome nor was there any analysis conducted to this effect. There was no consideration of using cognitive performance as a predictor but only as an outcome measure. The analyses described herein focus on the four cognitive task measures included in the Cogstate battery, as these data are available as z-scores wherein each individual's performance was normalized to that in a large healthy population. The tasks in this battery included a simple reaction time (RT) task, a choice RT task, a one-back working memory task, and a visual learning task. Moreover, given the conversion to z-score, a composite cognitive task performance score could be computed by averaging the z-scores for each of the four tasks in the battery. Analyses focused on the MADRS primary outcome of the study. Statistical analyses focused on the complete trial (incorporating both stage 1 and stage 2 outcomes), using the SPCD analytic approach reported in the original study and thus aligned could be compared with the original statistical analysis plan. Mixed models repeated measures (MMRM) were used to predict change from baseline (at 2, 4, and 6 weeks) in clinical scores and included covariates for group (i.e., poor versus good cognition as defined by a given measure of interest), time, time×group, time×baseline MADRS and baseline MADRS. These covariates likewise were used in the original study, and thus results here can be compared with the all-comer results reported previously. Statistical significance was thus assessed on the primary outcome (MADRS change from baseline at 6 weeks across the SPCD design). The change in MADRS scores in the all corner (i.e. original sample) analysis is shown in FIG. 1(A) for comparison to subgroups as defined by cognitive task performance.

To examine the effect of baseline cognition on clinical outcomes, individuals were split at the mean of the average z-score across all tasks (thus a cognition composite score). This generates two groupings, which were termed poor cognition (below mean, negative z scores) or good cognition (above mean, positive z scores). The poor cognition group can also be termed to be cognitively impaired. The composite score mean at which the cut-off between good and poor cognition groups was performed was at z=−0.32, which corresponded to the median of this patient group (and by a negative z score, denoted that being below the median for a matched healthy population). Notably, this mean is in line with expectations of moderately-impaired cognitive task performance in depression in general (12, 13).

Surprisingly, it was found that while the original all-comer analysis failed to find statistical significance on the primary MADRS outcome, a robust and statistically significant effect was found for the 80 mg arm versus placebo on change in MADRS scores in the poor cognition group using the cognitive composite score (SPCD analysis p=0.014; FIG. 1B). This corresponds to a drug-placebo effect size of Cohen's d=0.58, which is approximately double the typical d˜0.3 effect size of standard-of-care antidepressants in all-comer populations (31). Comparison of the 80 mg arm versus placebo in stage 1 of the trial also found statistical significance at week 2 (d=0.55, p=0.045), week 4 (d=0.7, p=0.01) and week 6 (d=0.59, p=0.03) with response being greater for 80 mg than placebo. Though the 40 mg group was visually intermediate in outcome to the 80 mg and placebo group, this effect was not significant (p=0.39; FIG. 1(B)). By contrast, no significant differences were found between either drug arm and placebo in the good cognition group (p=0.25, FIG. 1C). Thus, not only does cognitive task performance successfully differentially predict outcome between drug and placebo, this appears to be substantially stronger for the 80 mg treatment relative to 40 mg, which runs counter to expectations from prior treatment main effect analyses of the data as discussed above (11). This is also surprising since poor cognition was previously reported to be consistently predictive of poor response to SSRIs and SNRIs in multiple studies of depressed patients. Groves et al., Front. Psychiatry 9:382, 2018; Etkin et al., Neuropsychopharmacology 40(6):1332-1342, 2015.

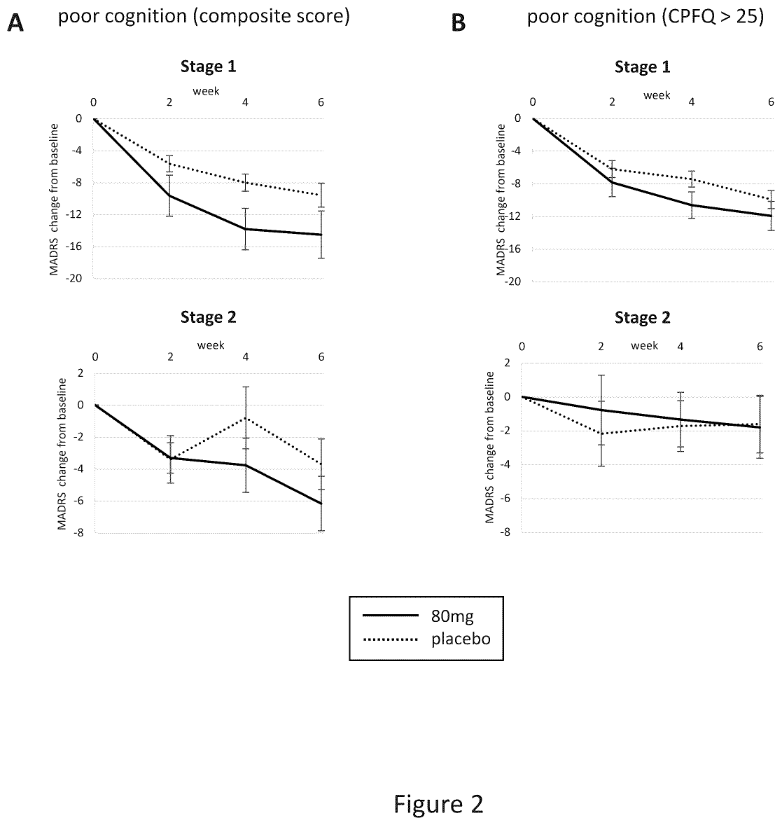

Whether poor cognition could be defined in an alternative manner was tested, namely by asking patients to rate their cognition on a self-report questionnaire. One such questionnaire is the Cognitive and Physical Functioning Questionnaire (CPFQ) (15). This scale is notable as it was featured in analyses for the antidepressant vortioxetine in its approval by both the FDA and the European Medicines Agency (EMA). In those analyses, poor subjective cognition was defined as a CPFQ score of >25, and thus whether cognitive impairment based on the CPFQ could identify individuals who preferentially benefit from treatment with 80 mg of NSI-189 was examined. As shown in FIG. 2, which contrasts poor cognition defined using the composite cognitive task performance score (as shown earlier) with that defined by the CPFQ, no difference is seen between treatment arms in patients with poor self-reported cognition. The difference between the 80 mg and placebo groups in CPFQ-defined poor cognition was comparable to that in the all-comer population. Thus, surprisingly, not only is poor objective cognition a predictor of better drug response, but this impairment must be measured through performance of cognitive tasks such as those used here. The objective measurement cannot be replaced by use of patient-reported assessment of their own cognitive symptoms. This furthermore did not depend on the cutoff used on the CPFQ to define poor cognition patients as multiple other cutoffs were used to define poor subjective cognition on the CPFQ and none were predictive of drug response.

While data from this Phase 2 trial demonstrated that patients with poor cognition respond better to NSI-189 than those with good cognition, the clinical sample was uninformative as to whether NSI-189 is more effective in poor cognition patients who had an insufficient response to a standard-of-care antidepressant. This is because only 18% of the patients receiving 80 mg of NSI-189 had an insufficient response to an adequate trial of an antidepressant according to the Antidepressant Treatment Response Questionnaire (ATRQ) in this Phase 2 sample.

### Example 2—Phase 2 Clinical Trial with NSI-189 as Monotherapy or Adjunctive Treatment in Depression

To determine whether poor cognition is a predictor of better response to 80 mg NSI-189 in both patients who were not receiving any other antidepressant treatments and patients who had an insufficient response to standard-of-care antidepressants, a large open-label Phase 2 clinical trial was conducted in which patients with MDD and/or PTSD were given 80 mg NSI-189 (as 40 mg BID) for up to 8 weeks. These patients also underwent a more comprehensive cognitive battery than in the prior Phase 2 study. As above, MMRM analyses were applied on MADRS change from baseline scores in MDD patients. This analysis included 90 patients, of whom 37 were receiving NSI-189 as monotherapy (i.e. no other concurrent antidepressant) and 53 were receiving it adjunctive to a standard-of-care antidepressant to which they had an insufficient response. The dose of the antidepressant (i.e. apart from NSI-189), if one was present at baseline, did not change during the trial. Of the overall sample, 61 had an insufficient response to at least one adequate trial of an antidepressant within the current episode, which included all patients receiving adjunctive NSI-189 as well as some receiving it as monotherapy. In this study, the current episode was determined by the either the prior two years, or the period within the prior two years since the last time at which two continuous months of euthymia occurred, whichever was shorter. Insufficient response to an antidepressant in the current episode was defined as less than a 50% reduction in symptoms (e.g., based on the total score on the MADRS or HDRS).

The overall sample using MMRMs that included core terms for time, baseline, group (i.e. poor versus good cognition as defined by a given measure of interest), time×group and time by baseline, as well as additional covariates for monotherapy/adjunctive use, time×monotherapy/adjunctive and time×group×monotherapy/adjunctive were first analyzed in order to control for the effects of NSI-189 treatment context within the overall analysis. All analyses used a z=−0.75 cutoff to identify poor cognition patients (with z-scores determined with reference to a separate healthy population) in order to ensure that poor cognition patients are impaired in those tasks. Approximately 40% of the MDD population in this study had cognition scores below the −0.75 cutoff, meaning that the poor cognition subpopulation makes up a significant and meaningful segment of the overall depression population. Moreover, as further detailed below, the relationship between degree of cognitive impairment (i.e. lower z-score) and better clinical outcome (i.e. greater MADRS change from baseline) is a continuous one, thereby supporting multiple cut-points to define poor cognition below z=0 (i.e., healthy mean score matched to patients' age and gender).

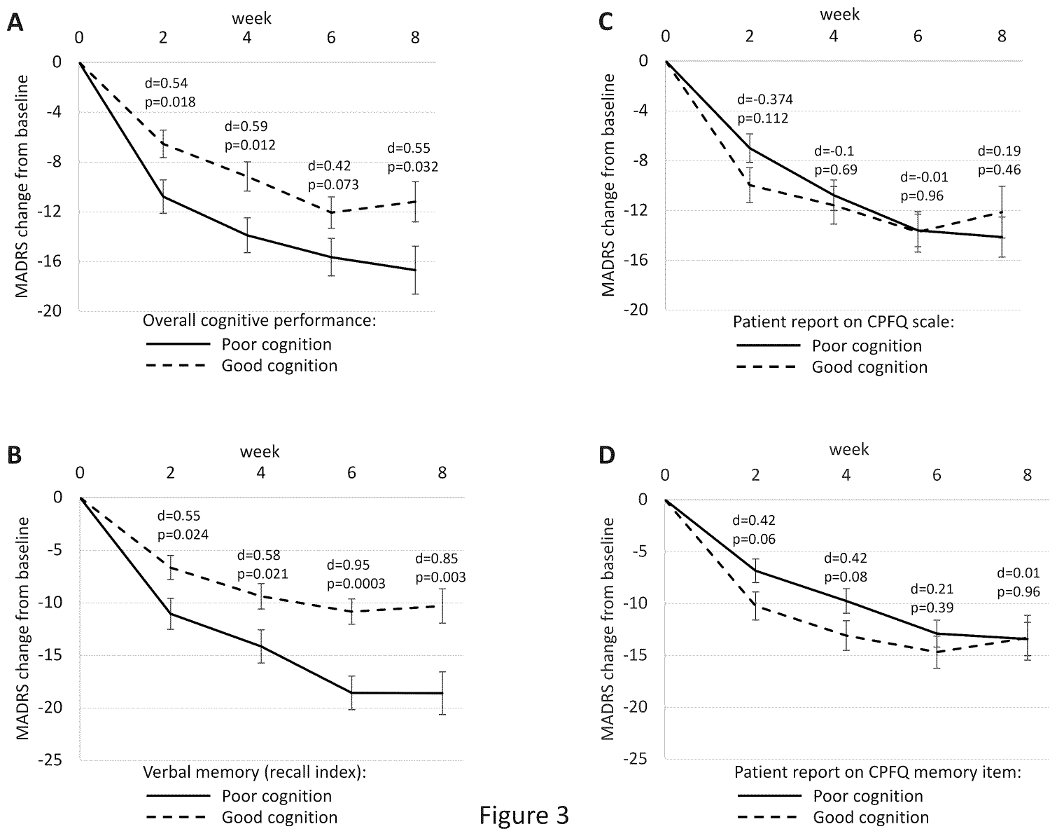

An overall cognitive performance variable comprised of measures of executive function (digit-symbol substitution task, Wisconsin card sorting task, trail making task, Corsi blocks, verbal fluency), information processing speed (flanker task, choice RT, simple RT) and verbal learning and/or memory was analyzed. As seen in FIG. 3(A), poor cognition patients had a significantly better improvement in depressive symptoms (reduction in the MADRS score) to treatment with NSI-189, further supporting the findings from the re-analysis of the prior Phase 2 trial where a general measure of poor cognition that encompassed executive function, processing speed and learning was used.

The ability of poor cognition to predict better outcome on depressive symptoms was most strongly carried by verbal learning and/or memory. Learning and/or memory was assessed with a word list task, in which patients are given the word lists on five learning trials, and then memory is assessed through recall of those word lists immediately after learning and again after an approximately 20 minute delay (including after inclusion of a new distractor list of words). This computer-implemented task is named VM-REACT (Verbal memory recall computerized test) and is further described in Naparstek et al., J Psychiatr Res., 2019, 114:170-177 (PMID 31096177). Poor recall of these word lists predicted significantly better outcome for MDD. This is seen in FIG. 3(B), where recall was quantified using the recall index, which averages both immediate and delayed recall in the verbal learning and/or memory task into a single memory recall measure. Additionally, poor immediate recall predicted significantly better outcome for MDD at weeks six and eight (Cohen's d=0.48, p=0.022; Cohen's d=0.56, p=0.010, respectively), and poor delayed recall predicted significantly better outcome for MDD at weeks two (Cohen's d=0.43, p=0.028), four (Cohen's d=0.40, p=0.045), six (Cohen's d=0.67, p=0.001) and eight (Cohen's d=0.61, p=0.004). Moreover, much as was found in the re-analysis of the prior Phase 2 study, patient reported cognitive impairment (as assessed on the CPFQ scale) did not predict outcome. Using the same definition of poor cognition as above (i.e. CPFQ >25), there were no significant differences between patients with self-reported poor versus good cognition (FIG. 3(C)). Likewise, taking just the memory recall item from the CPFQ, which is the subjective patient-reported measure most analogous to the recall index measure in FIG. 3(B), moderate or greater subjective diminishment of memory recall was not predictive of outcome with NSI-189 (FIG. 3(D)). Thus, it is critical to directly assess cognition using standardized behavioral tasks.

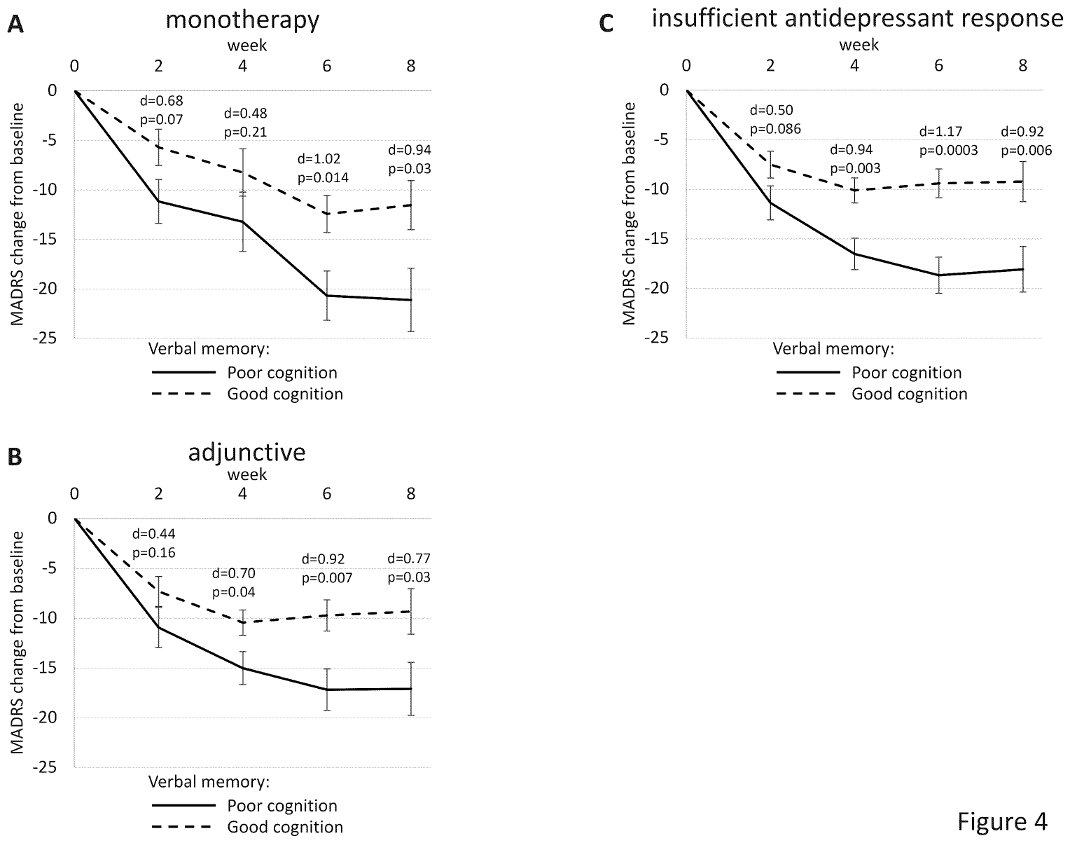

Prediction of NSI-189 outcome by poor cognition, as defined by recall index, separately for patients receiving the drug as monotherapy, as an adjunct to an antidepressant to which they had an insufficient response, or as a function of whether they have had an insufficient response to an antidepressant in the current episode (regardless of monotherapy or adjunctive use of NSI-189) was analyzed. As shown in FIGS. 4A and 4B, there was a similar magnitude and statistically significantly greater response to NSI-189 in poor cognition relative to good cognition patients in both the monotherapy and adjunctive treatment contexts. Thus, poor cognition predicts better depression symptom outcome with NSI-189 as adjunctive treatment in depression much as it does with NSI-189 as monotherapy—a conclusion not able to be drawn based on the prior Phase 2 study, which did not include adjunctive treatment. Likewise, patients were examined who had an insufficient response to at least one standard-of-care antidepressant treatment in the current depressive episode, a population that was too small in the prior Phase 2 study to be able to draw any conclusions from. As shown in FIG. 4C, poor cognition also predicted significantly better response to 80 mg NSI-189 in these patients. Thus, this new Phase 2 trial extended upon the prior Phase 2 trial by revealing novel evidence that NSI-189 is more effective for poor cognition patients that had an insufficient response to an antidepressant treatment in the current episode and either receive NSI-189 as monotherapy or adjunctively to that antidepressant. Additionally, this new trial extended upon the prior Phase 2 trial by showing that poor verbal memory recall as a measure of poor cognition (which was not previously assessed) is powerfully predictive of better response to NSI-189 regardless of whether the drug is used as a monotherapy or adjunctively to an antidepressant.

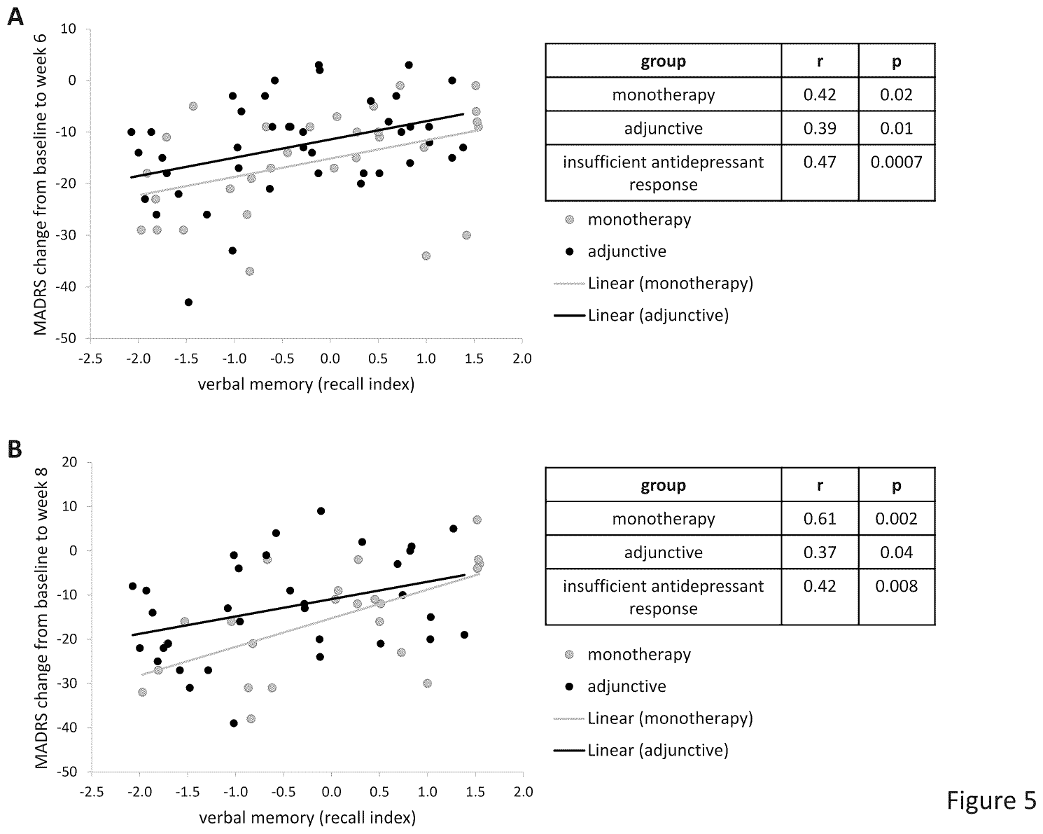

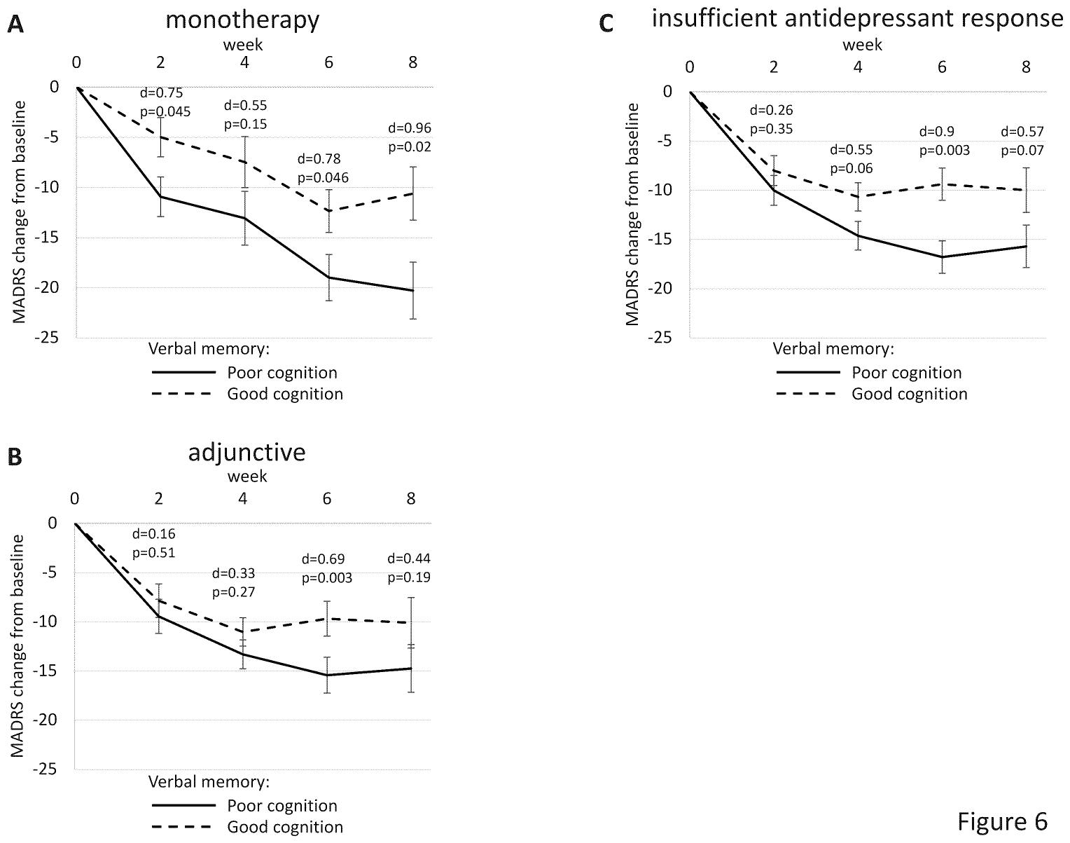

Moreover, though a cutoff of z=−0.75 was used to define poor cognition patients in the analyses above, the relationship between degree of cognitive impairment and degree of better depression treatment response is a continuous one. This is seen in correlations between change in MADRS scores from baseline to week 6 (FIG. 5(A)) or week 8 (FIG. 5(B)) and baseline learning and/or memory (recall index). As can be seen, there are significant correlations for each of these time points and each of the subpopulations examined (monotherapy, adjunctive, one or more insufficient treatment trials). Thus, poor cognition can be defined by any z-score on a cognitive metric below 0, which indicates the mean of a matched healthy population. The lower the z-score cutoff, the greater the expected NSI-189 clinical response of the thusly defined poor cognition patients. To illustrate this, FIG. 6 shows results similar to those in FIG. 4 whereby poor verbal memory predicts better outcome with NSI-189, however in FIG. 6 a cutoff of z=−0.418 is used instead of z=−0.75. The z=−0.418 cutoff corresponds to the median scores for patients in this study. As in FIG. 4, FIG. 6(A) shows patients receiving NSI-189 as monotherapy, FIG. 6(B) shows patients receiving NSI-189 adjunctively to an antidepressant to which they have had an insufficient response, and FIG. 6(C) shows patients receiving NSI-189 who had an insufficient response to an antidepressant in the current episode (which they may be still taking the antidepressant adjunctively or not).

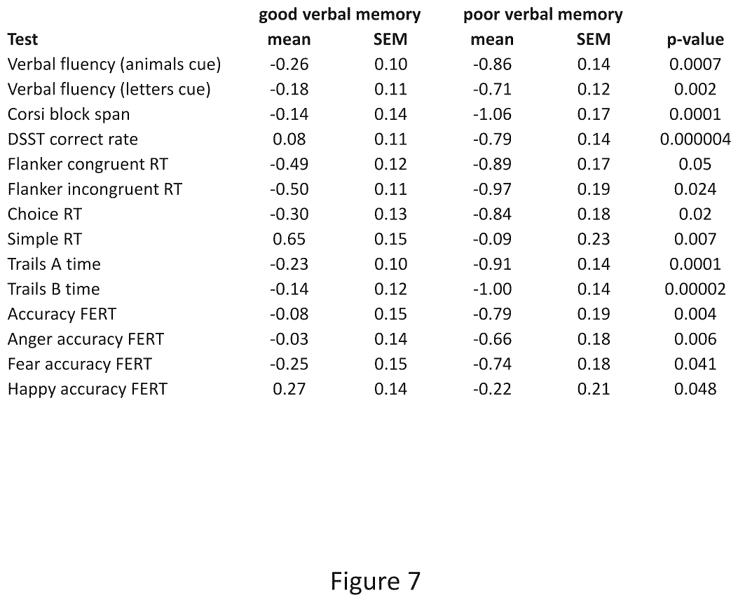

Though the analyses above have focused on using poor learning and/or memory to define poor cognition patients, doing so defines patients with impairments across a broad range of cognitive and behavioral measures. As seen in FIG. 7, poor cognition patients as defined by poor learning and/or memory (recall index) are impaired relative to good cognition patients in a wide variety of cognitive tasks, including: verbal fluency (number of words generated in response to a cue), working memory on a Corsi Block test (maximum number of sequenced actions correctly replicated in a row after watching a sequence performed), executive function and information processing speed on a digit-symbol substitution task (number of correct conversion of symbols to matched digits based on a given symbol-digit mapping), executive function and information processing speed on a flanker task (congruent reaction time), information processing speed on choice or simple reaction time tasks, information processing speed on the trails A task (completion time), interference resolution on a flanker task or trails B task (reaction time and completion time, respectively), and accuracy of emotion recognition in a face emotion recognition task (accuracy overall as well as for angry, fearful or happy expressions). Learning and/or memory, however, has been found to be a better predictor of depression treatment response with NSI-189 than these other objective cognitive measures.

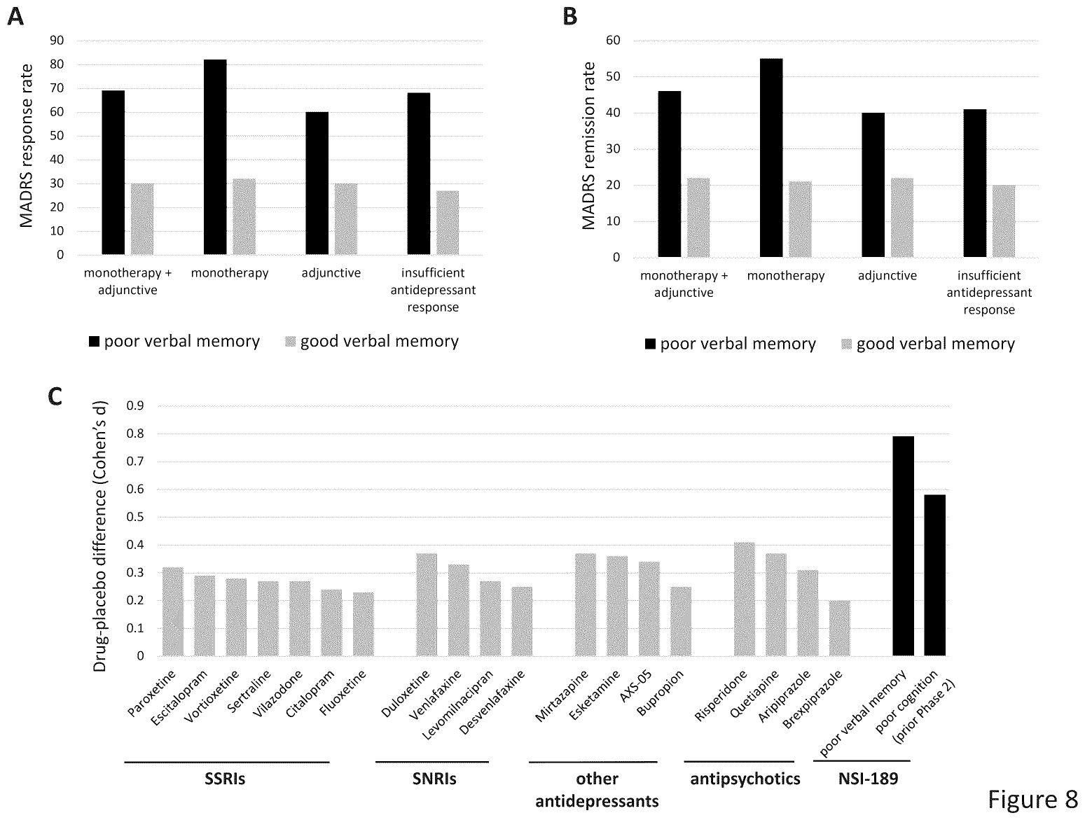

To further understand the clinical significance of the cognitive prediction of antidepressant outcome, plotted are response (FIG. 8(A)) and remission (FIG. 8(B)) rates amongst patients as a function of poor versus good learning and/or memory. As seen, response and remission rates are in poor learning and/or memory patients were consistently approximately double those of good learning and/or memory patients, regardless of how the clinical population was defined (i.e., overall depression, monotherapy, adjunctive, or one or more insufficient antidepressant treatment responses in the current episode). As an absolute level of response, the 60-82% response rates in poor learning and/or memory patients are strikingly higher than typical placebo response rates (˜35% for monotherapy and ˜20% for adjunctive therapy) (31-32), even considering the open label nature of this study. When considered from the context of ultimate clinical care, where a drug would be given in an open-label fashion, such high response rates serve as a contrast to current antidepressants where only one third to half of patients' response to treatment (33-34).

Next, it was sought to understand the contrast between response to NSI-189 in poor cognition/poor learning and/or memory patients, and the typical response to antidepressants or antipsychotics when used adjunctively to an antidepressant that had yielded an insufficient treatment response. To do so, the presumed drug-placebo difference for these patients was calculated based on two pieces of data: 1) the known all-comer drug placebo difference from the prior Phase 2 study (Cohen's d˜0.2), to which was added 2) the enrichment observed in the second Phase 2 study between the poor learning and/or memory subpopulation relative to the overall depression population (expressed as Cohen's d). Shown in FIG. 8(C) is the calculated drug-placebo Cohen's d for poor learning and memory patients in the second Phase 2 study (d=0.79), as well as the Cohen's d found in poor cognition patients in the prior Phase 2 study when averaging Stage 1 and Stage 2 responses (d=0.58). These striking effects of the drug could be contrasted to the effect sizes derived from a wide variety of antidepressants and antipsychotics (used adjunctively) (31-32 and 35-38), which were typically roughly half of the magnitude of the response to NSI-189. These results demonstrate the striking clinical significance of the identification of poor cognition as a predictor of better response to NSI-189 in depression. Moreover, poor cognition has been shown to predict worse response to placebo, creating the possibility that in poor cognition patients (39), this estimated drug-placebo difference could be even larger if the placebo response is further reduced in poor relative to good cognition patients. Likewise, poor cognition predicts poor response to standard-of-care antidepressants (e.g. SSRIs and SNRIs) (40), creating a striking contrast with NSI-189 to which response is greater in patients with poor cognition.

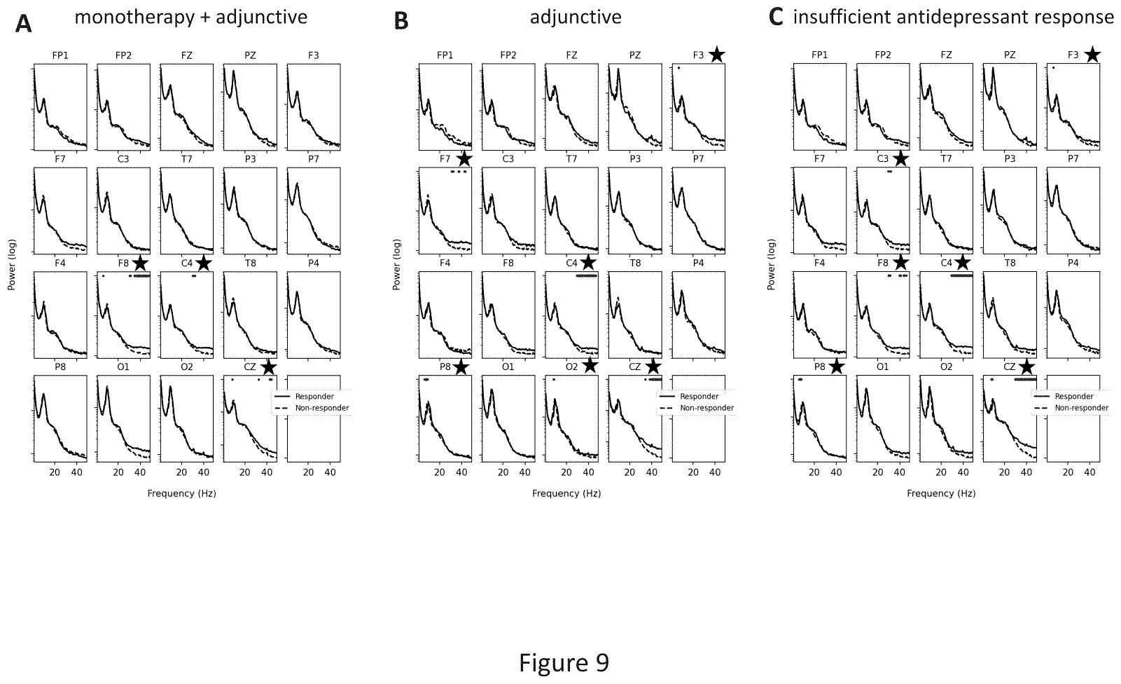

Next tested was whether signal derived from resting state electroencephalography (EEG) could predict treatment outcome with NSI-189. The power spectral density (PSD) distribution of the EEG data was examined. A schematic showing the EEG power spectral density is provided in FIGS. 1A and 1B in A. T. Hill et al., Dev. Cogn. Neurosci. 54:101076, 2022. As seen in FIG. 9(A) herein, PSDs at multiple channels indicate that depression symptom responders to NSI-189 have significantly higher power in the high beta frequency range (20-30 Hz) and low gamma range (31-50 Hz) (calculated over central electrodes). Additionally, responders have lower power in the alpha frequency range (8-12 Hz). The same pattern is seen in patients receiving adjunctive NSI-189 (FIG. 9(B)) and patients who have had an insufficient response to at least one antidepressant (FIG. 9(C)). This EEG pattern suggests that higher brain excitability (particularly the difference in power in the low gamma range) predicts better response to NSI-189.

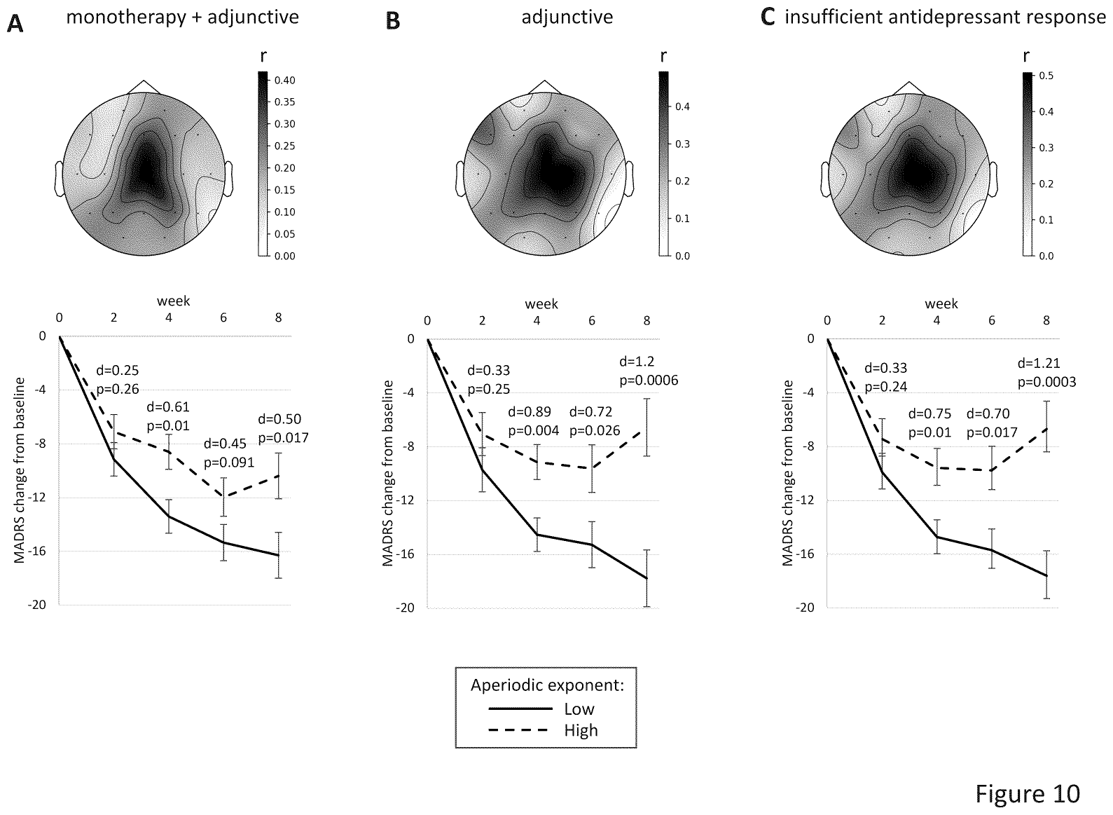

Another way of analyzing the PSD is through the observation that the background trend in the data can be quantified as a measure of background neural activity and has a 1/f relationship (where f is frequency). One way of quantifying this signal is termed the aperiodic exponent, which is estimated as the negative slope for the line of best fit over the 1-50 Hz range of the power spectral density in the log-log space (T. Donoghue, et al., Nat Neurosci, 23(12):1655-1665 December 2020). Channel-wise correlation of the aperiodic exponent with treatment outcome (as percentage change in MADRS scores from baseline) was examined. FIG. 10(A) (top panel) shows that a positive correlation whereby lower aperiodic exponent (i.e. flatter PSDs) (calculated over the central electrodes) predicts better depression treatment outcome (more negative percent MADRS change from baseline). This relationship was particularly strong for central-parietal electrode locations. The bottom panel shows an MMRM analysis splitting the patient population at the median and examining prediction of change in MADRS from baseline, demonstrating significantly better depression outcome in low aperiodic exponent patients relative to high aperiodic exponent patients. Similar relationships were seen between lower aperiodic exponent and better depression treatment outcome in patients receiving adjunctive NSI-189 (FIG. 10(B)), as well as in patients who had an insufficient response to an antidepressant in the current episode (FIG. 10(C)).

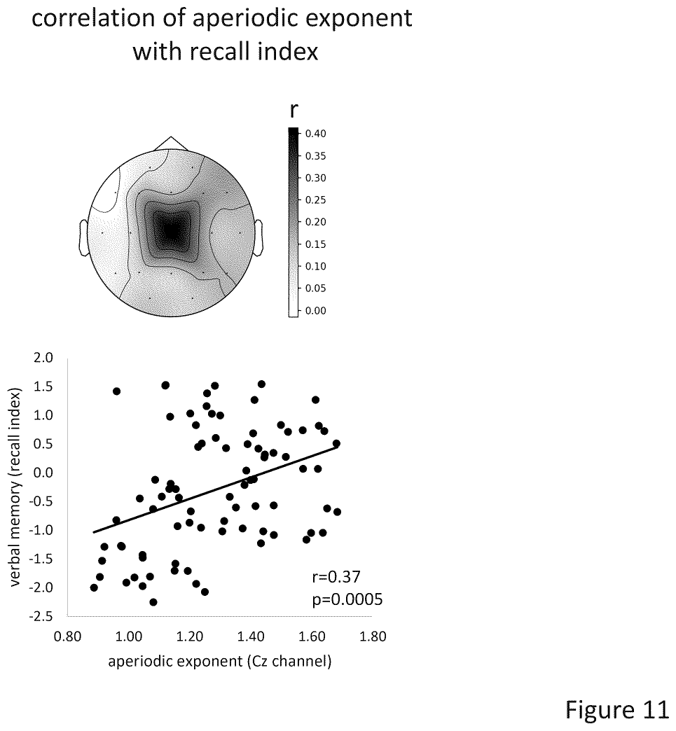

To understand the relationship between these EEG signals and memory recall index, either the channel-wise aperiodic exponent or power in the low gamma range were correlated with patients' recall index scores. As seen in FIG. 11, a larger aperiodic index (more steep power declines with higher frequencies in the PSD) was significantly correlated with greater recall index (better learning and/or memory). This is consistent with the findings above whereby better treatment outcome with NSI-189 is predicted by either a lower aperiodic exponent (more flat PSDs) and lower recall index scores, but furthermore relates the EEG and learning and/or memory signals to each other.

In summary, the findings from this Phase 2 study demonstrate that patients with either poor cognition (as measured by poor learning and memory recall) and/or greater brain excitability (as measured by lower aperiodic exponent or greater power in the low gamma range) have better depression treatment response to NSI-189, whether they received NSI-189 as a monotherapy, as an adjunct to an antidepressant to which they had an insufficient response, or in patients that have had an insufficient response in the current episode to an antidepressant drug regardless of whether NSI-189 is taken as a monotherapy or adjunctive to an antidepressant.

### Example 3—Phase 2 Clinical Trial with NSI-189 in PTSD

To determine whether poor cognition is a predictor of better response to 80 mg NSI-189 in PTSD, data was examined from patients with PTSD from the open-label Phase 2 clinical trial above in Example 2. These patients underwent the same cognitive battery as patients with depression. As above, MMRM analyses were applied on the Clinician Administered PTSD Scale for DSM-5 (CAPS-5) change from baseline scores in PTSD patients. This analysis included 84 patients. All patients received 40 mg NSI-189 BID, as the depressed patients had above.

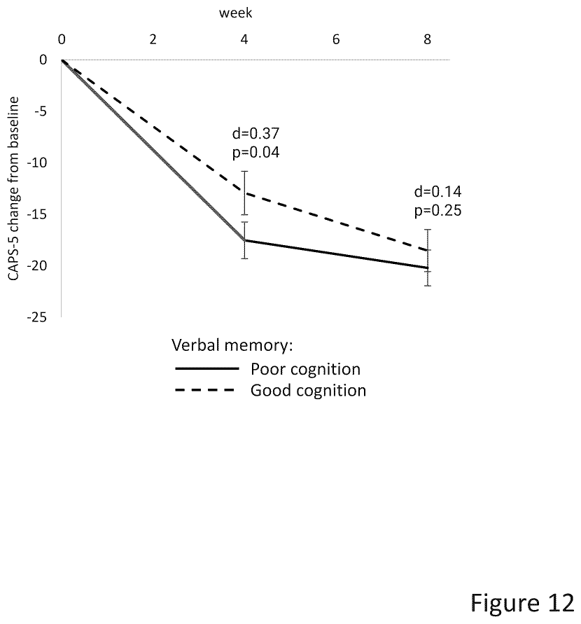

The overall sample using MMRMs that included core terms for time, baseline, group (i.e., poor versus good cognition as defined by a given measure of interest), time×group and time by baseline. The analyses shown in FIG. 12 used a z≤−0.5 cutoff on recall index scores in order to identify poor cognition patients (with z-scores determined with reference to a separate healthy population identically as for the depressed patients above). This cutoff ensured that poor cognition patients are impaired in this task. As seen in FIG. 12, a significant difference in outcome was observed at week 4, wherein patients with poor cognition had a significantly greater reduction in PTSD symptoms than those with good cognition.

### Example 4—Clinical Trial with NSI-189

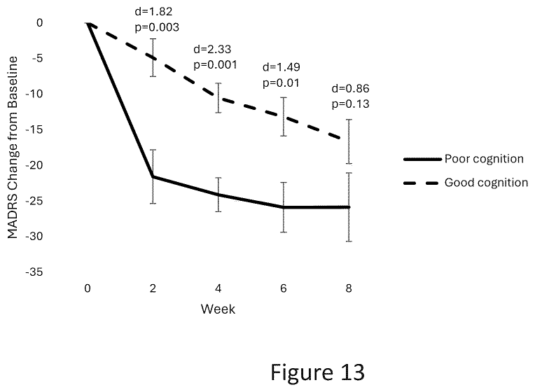

To examine the reproducibility of the ability of poor cognition to identify NSI-189 responders, an additional trial was run, which included 20 subjects with moderate to severe depression (n=6 classified as poor cognition based on poor verbal memory (z<−0.5) and n=14 classified as good cognition (z>−0.5). All patients were treated with 80 mg (40 mg BID) NSI-189 and assessed using MADRS scales for eight weeks. An analysis of Covariance (ANCOVA, controlling for baseline depression severity) was used to analyze the data at each week. Poor cognition patients demonstrated a large and statistically significantly greater improvement across weeks 2, 4, and 6, with a trend at week 8. The results are shown in FIG. 13. These data demonstrate in an independent patient cohort that poor cognition, as determined by poor verbal memory capacity, is a predictor of better antidepressant response to NSI-189.

### Example 5—Role of BDNF Signaling in the Effects of NSI-189

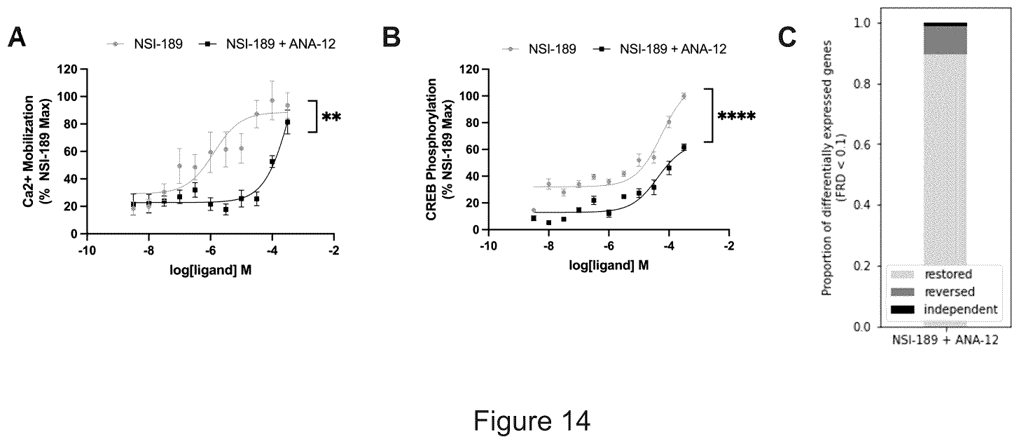

NSI-189 is a pro-neurogenic and pro-plasticity compound found to activate downstream effectors that lead to activation of BDNF, including intracellular calcium release and activation of multiple transcription factors. Moreover, the efficacy in activation of these pathways depends on the unhindered activity of the BDNF receptor TrkB. Specifically, NSI-189 leads to an immediate intracellular calcium mobilization in neural progenitor cells (NPCs) and rapid (2 hours post-treatment) phosphorylation of transcription factor CREB. These responses are drastically reduced when co-treating the cells with TrkB receptor blocker ANA-12. Furthermore, gene expression changes induced by NSI-189 in NPCs are restored or reversed with the addition of ANA-12. These data indicate that the TrkB-BDNF pathway is necessary for the molecular effects of NSI-189 in neuronal cells (FIG. 14).

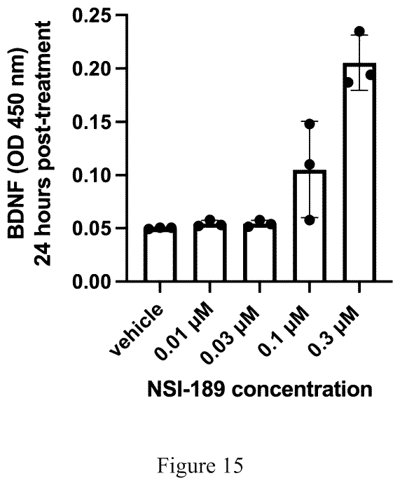

Additionally, NSI-189 was found to increase BDNF release in NPCs 24 hours post-treatment in a dose-dependent manner, displaying its pro-plasticity potential (FIG. 15).

### REFERENCES

1. Rodrigues R S, Paulo S L, Moreira J B, Tanqueiro S R, Sebastiao A M, Diogenes M J, Xapelli S. Adult Neural Stem Cells as Promising Targets in Psychiatric Disorders. Stem Cells Dev. 2020; 29:1099-1117.
2. Zarate C A, Jr., Singh J, Manji H K. Cellular plasticity cascades: targets for the development of novel therapeutics for bipolar disorder. Biol Psychiatry. 2006; 59:1006-1020.
3. Carli M, Aringhieri S, Kolachalam S, Longoni B, Grenno G, Rossi M, Gemignani A, Fornai F, Maggio R, Scarselli M. Is adult hippocampal neurogenesis really relevant for the treatment of psychiatric disorders? Curr Neuropharmacol. 2020.
4. Park S C. Neurogenesis and antidepressant action. Cell Tissue Res. 2019; 377:95-106.
5. Tunc-Ozcan E, Peng C Y, Zhu Y, Dunlop S R, Contractor A, Kessler J A. Activating newborn neurons suppresses depression and anxiety-like behaviors. Nat Commun. 2019; 10:3768.
6. Serafini G. Neuroplasticity and major depression, the role of modern antidepressant drugs. World J Psychiatry. 2012; 2:49-57.
7. Levy M J F, Boulle F, Steinbusch H W, van den Hove D L A, Kenis G, Lanfumey L. Neurotrophic factors and neuroplasticity pathways in the pathophysiology and treatment of depression. Psychopharmacology (Berl). 2018; 235:2195-2220.
8. Allen B D, Acharya M M, Lu C, Giedzinski E, Chmielewski N N, Quach D, Hefferan M, Johe K K, Limoli C L. Remediation of Radiation-Induced Cognitive Dysfunction through Oral Administration of the Neuroprotective Compound NSI-189. Radiat Res. 2018; 189:345-353.
9. Bauman M D, Schumann C M, Carlson E L, Taylor S L, Vazquez-Rosa E, Cintron-Perez C J, Shin M K, Williams N S, Pieper A A. Neuroprotective efficacy of P7C3 compounds in primate hippocampus. Transl Psychiatry. 2018; 8:202.
10. Fava M, Johe K, Ereshefsky L, Gertsik L G, English B A, Bilello J A, Thurmond L M, Johnstone J, Dickerson B C, Makris N, Hoeppner B B, Flynn M, Mischoulon D, Kinrys G, Freeman M P. A Phase 1B, randomized, double blind, placebo controlled, multiple-dose escalation study of NSI-189 phosphate, a neurogenic compound, in depressed patients. Mol Psychiatry. 2016; 21:1372-1380.
11. Papakostas G I, Johe K, Hand H, Drouillard A, Russo P, Kay G, Kashambwa R, Hoeppner B, Flynn M, Yeung A, Martinson M A, Fava M. A phase 2, double-blind, placebo-controlled study of NSI-189 phosphate, a neurogenic compound, among outpatients with major depressive disorder. Mol Psychiatry. 2020; 25:1569-1579.
12. Snyder H R. Major depressive disorder is associated with broad impairments on neuropsychological measures of executive function: a meta-analysis and review. Psychol Bull. 2013; 139:81-132.
13. Parkinson W L, Rehman Y, Rathbone M, Upadhye S. Performances on individual neurocognitive tests by people experiencing a current major depression episode: A systematic review and meta-analysis. J Affect Disord. 2020; 276:249-259.
14. Etkin A, Patenaude B, Song Y J, Usherwood T, Rekshan W, Schatzberg A F, Rush A J, Williams L M. A cognitive-emotional biomarker for predicting remission with antidepressant medications: a report from the iSPOT-D trial. Neuropsychopharmacology. 2015; 40:1332-1342.
15. Fava M, Iosifescu D V, Pedrelli P, Baer L. Reliability and validity of the Massachusetts general hospital cognitive and physical functioning questionnaire. Psychother Psychosom. 2009; 78:91-97.
16. Koenig A M, Bhalla R K, Butters M A. Cognitive functioning and late-life depression. J Int Neuropsychol Soc. 2014; 20:461-467.
17. Scott J C, Matt G E, Wrocklage K M, Crnich C, Jordan J, Southwick S M, Krystal J H, Schweinsburg B C. A quantitative meta-analysis of neurocognitive functioning in posttraumatic stress disorder. Psychol Bull. 2015; 141:105-140.
18. Hall M G, Hauson A O, Wollman S C, Allen K E, Connors E J, Stern M J, Kimmel C L, Stephan R A, Sarkissians S, Barlet B D, Grant I. Neuropsychological comparisons of cocaine versus methamphetamine users: A research synthesis and meta-analysis. Am J Drug Alcohol Abuse. 2018; 44:277-293.
19. Padilla-Coreano N, Canetta S, Mikofsky R M, Alway E, Passecker J, Myroshnychenko M V, Garcia-Garcia A L, Warren R, Teboul E, Blackman D R, Morton M P, Hupalo S, Tye K M, Kellendonk C, Kupferschmidt D A, Gordon J A. Hippocampal-Prefrontal Theta Transmission Regulates Avoidance Behavior. Neuron. 2019; 104:601-610 e604.
20. Egner T, Etkin A, Gale S, Hirsch J. Dissociable neural systems resolve conflict from emotional versus nonemotional distracters. Cereb Cortex 2008; 18:1475-84.
21. Xue S, Wang S, Kong X, Qiu J. Abnormal Neural Basis of Emotional Conflict Control in Treatment-Resistant Depression. Clin EEG Neurosci 2017; 48:103-10.
22. Xu M, Xu G, Yang Y. Neural Systems Underlying Emotional and Non-emotional Interference Processing: An ALE Meta-Analysis of Functional Neuroimaging Studies. Front Behav Neurosci 2016; 10:220.
23. Etkin A, Egner T, Peraza D M, Kandel E R, Hirsch J. Resolving emotional conflict: a role for the rostral anterior cingulate cortex in modulating activity in the amygdala. Neuron 2006; 51:871-82.
24. Maier M E, di Pellegrino G. Impaired conflict adaptation in an emotional task context following rostral anterior cingulate cortex lesions in humans. J Cogn Neurosci 2012; 24:2070-9.
25. Gyurak A, Patenaude B, Korgaonkar M S, Grieve S M, Williams L M, Etkin A. Frontoparietal Activation During Response Inhibition Predicts Remission to Antidepressants in Patients With Major Depression. Biol Psychiatry 2016; 79:274-81.
26. Williams L M, Korgaonkar M S, Song Y C, et al. Amygdala Reactivity to Emotional Faces in the Prediction of General and Medication-Specific Responses to Antidepressant Treatment in the Randomized iSPOT-D Trial. Neuropsychopharmacology 2015; 40:2398-408.
27. Johe et al., Ann Clin Psychiatry, 32(3):182-196 (2020).
28. Amos et al., J Clin Psychiatry, 79(2):17m11725, 2018.
29. Rush et al., N Engl J Med. 2006, 354:1231-1242.
30. Chandler et al., Validation of the Massachusetts General Hospital Antidepressant Treatment History Questionnaire (ATRQ). CNS Neurosci. Ther., 2010; 16:322-5.
31. Cipriani et al., Lancet, 2018 Apr. 7, 391(10128):1357-1366 (PMID 29477251).
32. Wen e., Braz J Med Biol Res, 2014 July, 47(7):605-16 (PMID 24919175).
33. Nelson, Am J Psychiatry, 2006 November, 163(11):1864-6 (PMID 17074931).
34. Warden et al., Curr Psychiatry Rep, 2007 December; 9 (6):449-59 (PMID 18221624).
35. Zhou et al., Int J Neuropsychopharmacol. 2015 May 25; 18(11):pyv060 (PMID 26012350).
36. Papakostas et al., J Clin Psychiatry. 2020 May 26; 81(4):19r12889 (PMID 32459407).
37. Kishi et al., Int J Neuropsychopharmacol., 2019 Nov. 1; 22(11):698-709 (PMID 31350882).
38. Iosifescu et al., J Clin Psychiatry, 2022 May 30; 83(4):21m14345 (PMID 35649167).
39. Ang et al., Psychol Med. 2020 Nov. 20; 1-9 (PMID 33213541).
40. Groves et al., Front Psychiatry, 2018 Aug. 28; 9:382 (PMID 30210368).
41. Levenberg et al., Gen Psychiatr. 2022, 35(4):e100760 (PMID 36035376).
42. Liu et al., Neuropharmacology, 2019, 144:337-344 (PMID 30408487).
43. Tajiri et al., J Cell Physiol., 2017, 232(10):2731-2740 (PMID 28181668).
44. Moliner et al., Nat Neurosci., 2023, 26(6):1032-1041 (PMID 37280397).
45. Banushi et al., Biology (Basel), 2023, 12(11):1380 (PMID 37997979).
46. Marton et al., Front Pharmacol., 2019, 10:193 (PMID 30890941).
47. Martínez-Turrillas et al., Neurosci Lett., 2006, 402(1-2):126-30 (PMID 16644117).
48. Calder et al., Neuropsychopharmacology, 2023, 48(1):104-112 (PMID 36123427).
49. Aleksandrova et al., Trends Pharmacol Sci., 2021, 42(11):929-942 (PMID 34565579).
50. Calabrese et al., Pharmacol Res., 2017, 121:59-69 (PMID 28442348).
51. Mozafari et al., Iran J Basic Med Sci., 2018, 724-730 (PMID 30140412).
52. Gordillo-Salas et al., Mol Neurobiol., 2020, 57(8):3498-3507 (PMID 32535760).
53. Destot-Wong et al., Neuropharmacology., 2009, 57(3):277-86 (PMID 19501111).
54. Lockhart et al., Eur J Pharmacol., 2007, 561(1-3):23-31 (PMID 17331496).
55. Mendez-David et al., Front Pharmacol., 2017, 8:462 (PMID 28769796).
56. Ward et al., J Psychopharmacol., 2020, 34(1):93-102 (PMID: 31766938).
57. Giralt et al., Neuropharmacology, 2017, 123:395-409 (PMID: 28603025).
58. Burgdorf et al., Int J Neuropsychopharmacol., 2022, 25(12):979-991 (PMID 35882204).
59. Donello et al., Int J Neuropsychopharmacol., 2019, 22(3):247-259 (PMID 30544218).
60. Burgdorf et al., Behav Brain Res., 2015, 294:177-85 (PMID 26210936).
61. Burgdorf et al., Neuroscience, 2015, 308:202-11 (PMID 26343295).
62. Zhang et al., Neuropharmacology, 2008, 55(7):1238-50 (PMID 18796308).

All publications, patents and patent applications cited herein are hereby incorporated by reference as if set forth in their entirety herein. While this invention has been described with reference to illustrative embodiments, this description is not intended to be construed in a limiting sense. Various modifications and combinations of illustrative embodiments, as well as other embodiments of the invention, will be apparent to persons skilled in the art upon reference to the description. It is therefore intended that the appended claims encompass such modifications and enhancements.

1. A method of treating major depressive disorder, post-traumatic stress disorder, or one or more symptoms thereof in a human patient having (a) objectively determined cognitive impairment or poor cognition, (b) objectively determined impaired learning and/or memory, (c) an electroencephalogram (EEG) exhibiting (i) a low aperiodic exponent, (ii) a high power in the low gamma range, (iii) a low power in the alpha frequency, (iv) low power at the centro-parietal electrodes in the theta frequencies, (v) low power at the centro-parietal electrodes in the alpha frequencies, (vi) low power at the frontal electrodes in the alpha frequencies, (vii) high aperiodic exponent at one or more posterior electrodes, or (viii) any combination of any of the foregoing, or (d) any combination of any of the foregoing, the method comprising administering to the patient an effective amount of a therapeutic agent, wherein the therapeutic agent is selected from (i) a 5-HT_{2A} agonist (including 5-HT_{2A} agonists that bind to a brain-derived neurotrophic factor (BDNF) receptor, e.g., a 5-HT_{2A} agonist that binds to the tropomyosin receptor kinase B (TrkB) receptor or low-affinity nerve growth factor receptor (p75NTR)), (ii) a therapeutic agent that promotes neuroplasticity by stimulation of the 5-HT_{2A} receptor, (iii) a therapeutic agent that releases BDNF by stimulation of the 5-HT_{2A} receptor (including those therapeutic agents that stimulate the 5-HT_{2A} receptor and bind to a TrkB receptor or p75NTR), (iv) an alpha-amino-3-hydroxy-5-methyl-4-isoxazole propionic acid (AMPA) positive allosteric modulator, (v) a therapeutic agent that promotes neuroplasticity by stimulation of the AMPA receptor, (vi) a therapeutic agent that releases BDNF by stimulation of the AMPA receptor, and (vii) an NMDA receptor positive allosteric modulator (such as a stinel compound).

2. The method of claim 1, wherein the human patient suffers from cognitive impairment or poor cognition as shown by one or more of a simple reaction time test, choice reaction time test, one back working memory task, and visual learning task.

3. The method of claim 1, wherein the human patient suffers from cognitive impairment or poor cognition as shown by a composite score which is at least partially based upon one or more results from a simple reaction time test, choice reaction time test, one back working memory task, or visual learning task.

4. The method of claim 1, wherein the patient suffers from reduced attention, memory, learning, working memory, or any combination of any of the foregoing.

5. The method of claim 1, wherein the patient suffers from major depressive disorder or one or more symptoms thereof and exhibits an electroencephalogram (EEG) with (i) a low aperiodic exponent, (ii) a high power in the low gamma range, (iii) a low power in the alpha frequency, or (iv) any combination of any of the foregoing.

6. The method of claim 1, wherein the patient has objectively determined impaired verbal learning and/or memory.

7. The method of claim 1, wherein the human patient suffers from impaired learning and/or memory as shown by VM-REACT (Verbal Memory REcAll Computerized Test), The Rey Auditory Verbal Learning Test, California Verbal Learning Test, California Verbal Learning Test—Short Form, California Verbal Learning Test—Children's Version, Hopkins Verbal Learning Test, Hopkins Verbal Learning Test—Revised, Philadelphia Verbal Learning Test, International Shopping List Test, Verbal section of the Repeatable Battery for the Assessment of Neuropsychological Status, Cerad Neuropsychological Assessment Battery Word List Task, Children's Auditory Verbal Learning Test, Children's Memory Scale, Bay Area Verbal Learning Test, Cogstate battery (which can include the following subtests: Behavioral Pattern Separation Object Test, Continuous Paired Associate Learning Test, Face Name Associative Memory Exam, Groton Maze Learning Test and its Delayed Recall and Delayed Reverse Recall versions, International Shopping List, One Card Learning Test), CANTAB (which can include the following subtests: Delayed Matching to Sample, Pattern Recognition Memory, Verbal Paired Associates, Paired Associates Learning, Verbal Recognition Memory), Penn Computerized Neurocognitive Battery (which can include the following subtests: Penn Word Memory Task, Penn Face Memory Task, Visual Object Learning Test), the NIH Toolbox and its subtests (Face Name Associative Memory Exam Test, Picture Sequence Memory Test, and Rey Auditory Verbal Learning Test), Neuropsychological Assessment Battery Memory Module, WHO/UCLA Auditory Verbal Learning Test, Repeatable Battery for the Assessment of Neuropsychological Status, Wide Range Assessment of Memory and Learning, Buschke Selective Reminding Test, Wechsler Memory Scale, Woodcock-Johnson Long Term Retrieval factor, Test of Memory and Learning, NEPSY, Brief Visuospatial Memory Test—Revised, Benton Visual Retention Test, Rey Osterreith Complex Figure Test, and any combination of any of the foregoing.

8. (canceled)

9. A method of treating major depressive disorder in a human patient having reduced information processing speed, attention, memory, learning, working memory, or any combination of any of the foregoing, comprising administering to the patient an effective amount of a therapeutic agent, wherein the therapeutic agent is selected from (i) a 5-HT_{2A} agonist (including 5-HT_{2A} agonists that bind to a BDNF receptor, e.g., a 5-HT_{2A} agonist that binds to the TrkB receptor or p75NTR), (ii) a therapeutic agent that promotes neuroplasticity by stimulation of the 5-HT_{2A} receptor, (iii) a therapeutic agent that releases BDNF by stimulation of the 5-HT_{2A} receptor (including those therapeutic agents that stimulate the 5-HT_{2A} receptor and bind to a TrkB receptor or p75NTR), (iv) an AMPA positive allosteric modulator, (v) a therapeutic agent that promotes neuroplasticity by stimulation of the AMPA receptor, (vi) a therapeutic agent that releases BDNF by stimulation of the AMPA receptor, and (vii) an NMDA receptor positive allosteric modulator (such as a stinel compound).

10. The method of claim 1, wherein the patient suffers from anhedonia, suicidality, or both.

11. The method of claim 1, wherein the patient suffers from reduced information processing speed.

12. A method of treating one or more symptoms selected from depressive symptoms, anhedonia, loss of interest, avolition, diminished emotional expression, inability to feel, amotivation, apathy, slow thinking, psychomotor retardation, lassitude, or any combination of any of the foregoing in a human patient suffering from post-traumatic stress disorder, bipolar depression, substance use disorder or schizophrenia, where the patient has (a) objectively determined cognitive impairment or poor cognition, (b) objectively determined impaired learning and/or memory, (c) an electroencephalogram (EEG) exhibiting (i) a low aperiodic exponent, (ii) a high power in the low gamma range, (iii) a low power in the alpha frequency, (iv) low power at the centro-parietal electrodes in the theta frequencies, (v) low power at the centro-parietal electrodes in the alpha frequencies, (vi) low power at the frontal electrodes in the alpha frequencies, (vii) high aperiodic exponent at one or more posterior electrodes, or (viii) any combination of any of the foregoing, or (d) any combination of any of the foregoing, the method comprising administering to the patient an effective amount of a therapeutic agent, wherein the therapeutic agent is selected from (i) a 5-HT_{2A} agonist (including 5-HT_{2A} agonists that bind to a BDNF receptor, e.g., a 5-HT_{2A} agonist that binds to the TrkB receptor or p75NTR), (ii) a therapeutic agent that promotes neuroplasticity by stimulation of the 5-HT_{2A} receptor, (iii) a therapeutic agent that releases BDNF by stimulation of the 5-HT_{2A} receptor (including those therapeutic agents that stimulate the 5-HT_{2A} receptor and bind to a TrkB receptor or p75NTR), (iv) an AMPA positive allosteric modulator, (v) a therapeutic agent that promotes neuroplasticity by stimulation of the AMPA receptor, (vi) a therapeutic agent that releases BDNF by stimulation of the AMPA receptor, and (vii) an NMDA receptor positive allosteric modulator (such as a stinel compound).

13. (canceled)

14. The method of claim 12, wherein the patient is concurrently treated with one or more antipsychotic medications, mood stabilizers, or any combination of any of the foregoing.

15-34. (canceled)

35. The method of claim 1, wherein the patient has impaired learning and/or memory as objectively determined by poor immediate recall in a verbal memory test.

36. The method of claim 1, wherein the patient has impaired learning and/or memory as objectively determined by poor delayed recall in a verbal memory test.

37. The method of claim 1, wherein the patient is not concurrently treated with a second antidepressant medication.

38. The method of claim 1, wherein the patient is concurrently treated with a second antidepressant medication.

39. The method of claim 1, wherein prior to treatment with the therapeutic agent, the patient had an insufficient response to an antidepressant other than the therapeutic agent.

40. The method of claim 1, wherein the patient was, prior to treatment with the therapeutic agent, treated with one or more antidepressants and continues treatment with the one or more antidepressants during treatment with the therapeutic agent.

41. The method of claim 39, wherein the one or more antidepressants do not include a monoamine oxidase inhibitor (MAOI) or a tricyclic antidepressant.

42. The method of claim 39, wherein the one or more antidepressants are selected from serotonin reuptake inhibitors, serotonin and norepinephrine reuptake inhibitors, mirtazapine, bupropion, and any combination of any of the foregoing.

43-52. (canceled)
